(19) United States
(12) Patent Application Publication
(10) Pub. No.: US 2014/0142352 A1
(43) Pub. Date: May 22, 2014

(54) BUTANOL PURIFICATION

(71) Applicant: BUTAMAX ADVANCED BIOFUELS LLC, Wilmington, DE (US)

(72) Inventors: MICHAEL DAUNER, Wilmington, DE (US); Robert Dicosimo, Chadds Ford, PA (US); Steven D. Doig, Wilmington, DE (US); Adam David Henry, Cottingham (GB); Joseph J. Zaher, Newark, DE (US)

(73) Assignee: BUTAMAX ADVANCED BIOFUELS LLC, Wilmington, DE (US)

(21) Appl. No.: 14/084,881

(22) Filed: Nov. 20, 2013

Related U.S. Application Data
(60) Provisional application No. 61/728,400, filed on Nov. 20, 2012.

(51) Int. Cl. C12P 7/16; C07C 31/12

(52) U.S. Cl. CPC C12P 7/16; C07C 31/12
USPC 568/913; 435/160; 568/840

(57) ABSTRACT

Provided herein are processes for adjusting a fermentation medium to reduce the activity of one or more carboxylic acids. The processes comprise (a) providing a recombinant microorganism comprising an engineered butanol biosynthetic pathway, (b) contacting the recombinant microorganism with a fermentation medium whereby butanol is produced and wherein the fermentation medium comprises one or more carboxylic acids, and (c) adjusting the fermentation medium to reduce the activity of the one or more carboxylic acids. Also provided are processes for reducing the activity of one or more carboxylic acids in a feed. The processes comprise (a) providing a feed from a fermentation vessel, wherein the feed comprises a composition produced by a recombinant microorganism comprising an engineered butanol biosynthetic pathway, wherein the composition comprises butanol, water, and one or more carboxylic acids; and (b) adjusting the feed, wherein adjusting the feed reduces the activity of the one or more carboxylic acids.

### CROSS REFERENCE TO RELATED APPLICATION

This application claims benefit of priority from U.S. Provisional Application No. 61/728,400, filed Nov. 20, 2012, which is hereby incorporated by reference in its entirety.

### FIELD OF INVENTION

The present invention relates to processes for reducing the activity of one or more carboxylic acids. More specifically, the invention relates to processes for reducing the activity of one or more carboxylic acids in a butanol based composition, wherein the butanol based composition is produced by a recombinant microorganism in a fermentation medium.

### BACKGROUND

Butanol is an important industrial chemical with a variety of applications, including use as a fuel additive, as a feedstock chemical in the plastics industry, and as a food-grade extractant in the food and flavor industry. Accordingly there is a high demand for butanol, as well as for efficient and environmentally friendly production methods. One such environmentally friendly production method includes the production of butanol utilizing fermentation by microorganisms. During the fermentation and subsequent purification steps, the butanol produced can comprise one or more carboxylic acids that are deleterious in fuels prepared by blending the butanol with gasoline, one or more components of gasoline or other hydrocarbon-based fuels. The methods provided herein can reduce the activity of the one or more carboxylic acids in the butanol compositions produced by fermentation methods, thereby making the butanol more acceptable for end use applications to meet industry or end-user specifications.

The present invention satisfies the need to reduce the activity of the one or more carboxylic acids in bio-produced butanol based compositions.

### BRIEF SUMMARY OF THE INVENTION

Provided herein are processes for adjusting a fermentation medium to reduce the activity of one or more carboxylic acids. The processes comprise (a) providing a recombinant microorganism comprising an engineered butanol biosynthetic pathway, (b) contacting the recombinant microorganism with a fermentation medium whereby butanol is produced and wherein the fermentation medium comprises one or more carboxylic acids, and (c) adjusting the fermentation medium to reduce the activity of the one or more carboxylic acids. The processes can further comprise a distillation step (d), wherein the distillation step results in the isolation of butanol. Optionally, reducing the activity of the one or more carboxylic acids may include neutralizing (e.g., increasing the pH), chemically modifying, destroying, complexing, and/or sequestering the one or more carboxylic acids.

Adjusting the fermentation medium can, for example, include contacting the fermentation medium with an agent. The contacting step can, for example, occur during the fermentation process (e.g., during a propagation phase and/or a production phase). Optionally, adjusting the fermentation medium further comprises distilling the fermentation medium, whereby the distillation step results in the isolation of the butanol from the composition. Optionally, the contacting step occurs after fermentation and prior to distillation. By way of an example, the contacting step can occur in one or more beer wells or in one or more external extractor units (e.g., a siphon, a decanter, a centrifuge, a gravity settler, a phase splitter, a mixer-settler, a column extractor, a centrifugal extractor, a hydrocyclone spray tower, or combinations thereof) prior to distillation. Optionally, the contacting step occurs during distillation. Optionally, the contacting step occurs during fermentation, prior to distillation, and during distillation. The distillation step can, for example, comprise a distillation unit comprising at least one distillation column and at least one decanter vessel. By way of an example, the contacting step can occur in the decanter vessel during distillation. Optionally, the adjusting step occurs after the distillation step. The adjusting step can occur in a recycle stream from the distillation unit. By way of an example, the recycle stream is provided to an anaerobic digester, which contains an agent (i.e., a microorganism) capable of degrading or destroying the carboxylic acids in the recycle stream.

Also provided are processes for reducing the activity of one or more carboxylic acids in a feed. The processes comprise (a) providing a feed from a fermentation vessel, wherein the feed comprises a composition produced by a recombinant microorganism comprising an engineered butanol biosynthetic pathway, wherein the composition comprises butanol, water, and one or more carboxylic acids; and (b) adjusting the feed, wherein adjusting the feed reduces the activity of the one or more carboxylic acids. Optionally, adjusting the feed can comprise contacting the feed with an agent, wherein the agent reduces the activity of the one or more carboxylic acids. The processes can further comprise a distillation step (c), wherein the distillation step results in the isolation of the butanol from the composition.

Optionally, the adjusting step (b) occurs prior to, during, or prior to and during the distillation step (c). The distillation step (c) can comprise a distillation unit comprising at least one distillation column and at least one decanter vessel. In the event that the adjusting step (b) occurs prior to the distillation step (c), the adjusting step can, for example, occur in one or more beer wells (e.g., an agent can be added to the one or more beer wells). In the event that the adjusting step (b) occurs during the distillation step (c), the adjusting step can, for example, occur in the at least one decanter vessel (e.g., an agent can be added to the decanter vessel). Optionally, the adjusting step (b) occurs after the distillation step (c). The adjusting step can occur in a recycle stream from the distillation step. By way of an example, the adjusting step can comprise providing the recycle stream to an anaerobic digester, which contains an agent (i.e., a microorganism) capable of degrading or destroying the carboxylic acids in the feed.

Optionally, the carboxylic acid is selected from, but not limited to, the group consisting of butyric acid, valeric acid, propanoic acid, formic acid, and acetic acid. The carboxylic acid can be butyric acid. The butyric acid can, for example, be isobutyric acid.

Optionally, the agent can be selected from the group consisting of a sequestering agent, a complexation agent, a neutralizing agent, a modifying agent, and a destructive agent. The neutralizing agent can, for example, be an agent that increases the pH of the composition. Optionally, the neutralizing agent is a base. The base can be selected from, but is not limited to, the group consisting of calcium oxide, calcium hydroxide, calcium carbonate, sodium hydroxide, sodium carbonate, sodium phosphate, sodium ethoxide, potassium hydroxide, potassium carbonate, potassium phosphate, magnesium hydroxide, ammonium hydroxide, and combinations thereof. Optionally, the neutralizing agent can be selected from, but is not limited to, the group consisting of urea, fatty amines, anhydrous ammonia, and ion exchange resin. Optionally, the agent can be a modifying agent. The modifying agent can, for example, be an esterifying agent.

Also provided herein are compositions produced by the processes described herein, wherein the composition comprises less than 1 weight percent carboxylic acid. The composition can comprise less than 0.10 weight percent carboxylic acid, preferably less than 0.01 weight percent carboxylic acid, and most preferably less than 0.001 weight percent carboxylic acid.

### BRIEF DESCRIPTION OF FIGURES

FIG. 1 shows a schematic of a butanol production facility.

FIG. 2 shows a schematic of the fermentation process.

FIG. 3 shows a schematic of a distillation unit. The schematic illustrates the addition of the agent prior to distillation and during distillation.

FIG. 4 shows a graph demonstrating effective titers in aqueous media of consumed glucose (GLC), produced isobutanol (ISO), and produced glycerol (GLY). Error bars indicate an assumed standard deviation of the measurement of +/−5%.

FIG. 5 shows a graph of the aqueous concentrations of analyzed organic acids in corn mash in cultures with different extractant mixtures. Error bars indicate an assumed standard deviation of the measurement of +/−5%. Abbreviations: PYR: pyruvic acid, KIV: ketoisovaleric acid, DHIV: dihydroxyisovaleric acid, ACA: acetic acid, IBA: isobutyric acid, LAC: lactic acid, SUC: succinic acid.

FIG. 6 shows a graph of the observed aqueous concentrations of organic acids in the corn mash fermentation of an isobutanologen as a percentage of isobutanol produced. The lower concentrations of organic acids in the OA:TOA mixtures as compared to the OA extractant indicates TOA complexes deprotonated organic acids and sequesters the deprotonated organic acids in the extractant mixture.

### DETAILED DESCRIPTION OF THE INVENTION

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference, unless only specific sections of patents or patent publications are indicated to be incorporated by reference.

In order to further define this invention, the following terms and definitions are herein provided.

As used herein, the terms “comprises,” “comprising,” “includes,” “including,” “has,” “having,” “contains,” or “containing,” or any variation thereof, will be understood to imply the inclusion of a stated integer or group of integers but not the exclusion of any other integer or group of integers. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances, i.e., occurrences of the element or component. Therefore “a” or “an” should be read to include one or at least one, and the singular word form of the element or component also includes the plural unless the number is obviously meant to be singular.

The term “invention” or “present invention” as used herein, is a non-limiting term and is not intended to refer to any single embodiment of the particular invention but encompasses all possible embodiments as described in the application.

As used herein, the term “about” modifying the quantity of an ingredient or reactant of the invention employed refers to variation in the numerical quantity that can occur, for example, through typical measuring and liquid handling procedures used for making concentrates or solutions in the real world; through inadvertent error in these procedures; through differences in the manufacture, source, or purity of the ingredients employed to make the compositions or to carry out the methods; and the like. The term “about” also encompasses amounts that differ due to different equilibrium conditions for a composition resulting from a particular initial mixture. Whether or not modified by the term “about,” the claims include equivalents to the quantities. In one embodiment, the term “about” means within 10% of the reported numerical value, alternatively within 5% of the reported numerical value.

The term “aerobic conditions” as used herein, means growth conditions in the presence of oxygen.

The term “microaerobic conditions” as used herein, means growth conditions with low levels of oxygen (i.e., below normal atmospheric oxygen levels).

The term “anaerobic conditions” as used herein, means growth conditions in the absence of oxygen.

“Biomass” as used herein, refers to a natural product comprising hydrolysable polysaccharides that provide fermentable sugars, including any sugars and starch derived from natural resources such as corn, sugar cane, wheat, cellulosic or lignocellulosic material and materials comprising cellulose, hemicellulose, lignin, starch, oligosaccharides, disaccharides and/or monosaccharides, and mixtures thereof. Biomass may also comprise additional components, such as protein and/or lipids. Biomass may be derived from a single source, or biomass can comprise a mixture derived from more than one source; for example biomass may comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, waste sugars, wood and forestry waste. Examples of biomass include, but are not limited to, corn grain, corn cobs, crop residues such as corn husks, corn stover, grasses, wheat, rye, wheat straw, barley, barley straw, hay, rice straw, switchgrass, waste paper, sugar cane bagasse, sorghum, soy, whey, whey permeate, components obtained from milling of grains, trees, branches, roots, leaves, wood chips, sawdust, shrubs and bushes, vegetables, fruits, flowers, animal manure, and mixtures thereof. For example, mash, juice, molasses, or hydrolysate may be formed from biomass by any processing known in the art for processing the biomass for purposes of fermentation, such as by milling, treating and/or liquefying and comprises fermentable sugar and may comprises an amount of water. For example, corn may be processed via wet mill or dry mill and subsequently liquefied to produce mash. Cellulosic and/or lignocellulosic biomass may be processed to obtain a hydrolysate containing fermentable sugars by any method known to one skilled in the art (see, e.g., U.S. Patent Application Publication No. 2007/0031918, which is herein incorporated by reference). Enzymatic saccharification of cellulosic and/or lignocellulosic biomass makes use of an enzyme consortium for breaking down cellulose and hemicellulose to produce a hydrolysate containing sugars including glucose, xylose, and arabinose. Saccharification enzymes suitable for cellulosic and/or lignocellulosic biomass are reviewed in Lynd et al., Microbiol. Mol. Biol. Rev. 66:506-77 (2002).

The term “biomass” as used herein, in some instances refers to the mass of the culture, e.g., the amount of recombinant microorganisms, typically provided in units of grams per liter (g/l) dry cell weight (dcw).

“Biomass yield” as used herein, refers to the ratio of microorganism biomass produced (i.e., cell biomass production) to carbon substrate consumed.

“Biofuel” or “biofuel product” as used herein, refers to a fuel derived from a biological process, for example, but not limited to, fermentation.

“Butanol” as used herein, refers to the butanol isomers 1-butanol (1-BuOH), 2-butanol (2-BuOH), tert-butanol (t-BuOH), and/or isobutanol (iBuOH or i-BuOH, also known as 2-methyl-1-propanol), either individually or as mixtures thereof. From time to time, as used herein the terms “biobutanol” and “bio-produced butanol” may be used synonymously with “butanol.”

Uses for butanol can include, but are not limited to, fuels (e.g., biofuels), a fuel additive, an alcohol used for the production of esters that can be used as diesel or biodiesel fuel, as a chemical in the plastics industry, an ingredient in formulated products such as cosmetics, and a chemical intermediate. Butanol may also be used as a solvent for paints, coatings, varnishes, resins, gums, dyes, fats, waxes, resins, shellac, rubbers, and alkaloids.

As used herein, the term “bio-produced” means that the molecule (e.g., butanol) is produced from a renewable source (e.g., the molecule can be produced during a fermentation process from a renewable feedstock). Thus, for example, bio-produced isobutanol can be isobutanol produced by a fermentation process from a renewable feedstock. Molecules produced from a renewable source can further be defined by the ¹⁴C/¹²C isotope ratio. A ¹⁴C/¹²C isotope ratio in range of from 1:0 to greater than 0:1 indicates a bio-produced molecule, whereas a ratio of 0:1 indicates that the molecule is fossil derived.

“Product alcohol” as used herein, refers to any alcohol that can be produced by a microorganism in a fermentation process that utilizes biomass as a source of fermentable carbon substrate. Product alcohols include, but are not limited to, C₁ to C₈ alkyl alcohols, and mixtures thereof. In some embodiments, the product alcohols are C₂ to C₈ alkyl alcohols. In other embodiments, the product alcohols are C₂ to C₅ alkyl alcohols. It will be appreciated that C₁ to C₈ alkyl alcohols include, but are not limited to, methanol, ethanol, propanol, butanol, pentanol, and mixtures thereof. Likewise C₂ to C₈ alkyl alcohols include, but are not limited to, ethanol, propanol, butanol, and pentanol. “Alcohol” is also used herein with reference to a product alcohol.

The term “effective titer” as used herein, refers to the total amount of a particular alcohol (e.g., butanol) produced by fermentation or alcohol equivalent of the alcohol ester produced by alcohol esterification per liter of fermentation medium. For example, the effective titer of butanol in a unit of volume of a fermentation includes: (i) the amount of butanol in the fermentation medium; (ii) the amount of butanol recovered from the organic extractant; (iii) the amount of butanol recovered from the gas phase, if gas stripping is used; and (iv) the alcohol equivalent of the butyl ester in either the organic or aqueous phase.

The term “effective rate” as used herein, is the effective titer divided by the fermentation time.

The term “effective yield” as used herein, is the total grams of product alcohol produced per gram of glucose consumed.

“In Situ Product Removal” (ISPR) as used herein, means the selective removal of a fermentation product from a biological process such as fermentation to control the product concentration as the product is produced.

“Fermentable carbon source” or “fermentable carbon substrate” as used herein, means a carbon source capable of being metabolized by the microorganisms disclosed herein for the production of fermentative alcohol. Suitable fermentable carbon sources include, but are not limited to, monosaccharides such as glucose or fructose; disaccharides such as lactose or sucrose; oligosaccharides; polysaccharides such as starch or cellulose; C5 sugars such as xylose and arabinose; one carbon substrates including methane; amino acids; and mixtures thereof.

“Feedstock” as used herein, means a feed in a fermentation process, the feed containing a fermentable carbon source with or without undissolved solids, and where applicable, the feed containing the fermentable carbon source before or after the fermentable carbon source has been liberated from starch or obtained from the breakdown of complex sugars by further processing such as by liquefaction, saccharification, or other process. Feedstock includes or is derived from a biomass. Suitable feedstocks include, but are not limited to, rye, wheat, corn, corn mash, cane, cane mash, barley, cellulosic material, lignocellulosic material, or mixtures thereof. Where reference is made to “feedstock oil,” it will be appreciated that the term encompasses the oil produced from a given feedstock.

“Sugar” as used herein, refers to oligosaccharides, disaccharides, monosaccharides, and/or mixtures thereof. The term “saccharide” also includes carbohydrates including starches, dextrans, glycogens, cellulose, pentosans, as well as sugars.

“Fermentable sugar” as used herein, refers to one or more sugars capable of being metabolized by the microorganisms disclosed herein for the production of fermentative alcohol.

“Undissolved solids” as used herein, means non-fermentable portions of feedstock, for example, germ, fiber, and gluten. For example, the non-fermentable portions of feedstock include the portion of feedstock that remains as solids and can absorb liquid from the fermentation broth.

“Fermentation medium” as used herein, means the mixture of water, sugars (fermentable carbon substrates), dissolved solids (if present), optionally microorganisms producing alcohol, product alcohol, and all other constituents of the material in which product alcohol is made by the reaction of fermentable carbon substrates (e.g., sugars) to alcohol, water, and carbon dioxide (CO₂) by the microorganisms present. From time to time, as used herein the term “fermentation broth” and “fermented mixture” can be used synonymously with “fermentation medium.”

The term “biphasic fermentation medium” as used herein, refers to a two-phase growth medium comprising a fermentation medium (i.e., aqueous phase) and a suitable amount of a water immiscible organic extractant.

The term “propagation phase” or “growth phase” as used herein refers to the process steps during which the recombinant microorganism (e.g., yeast) biomass is produced.

The term “production phase” as used herein refers to the fermentation or other process steps during which a desired fermentation product, including, but not limited to butanol, isobutanol, 1-butanol, and/or 2-butanol is produced.

“Seed train” as used herein, refers to a series of biomass amplification stages of the recombinant microorganism prior to introduction into the propagation vessel. A seed train can comprise multiple vessels, wherein each vessel is smaller than or equal to the vessel into which the recombinant microorganism is introduced (e.g., a seed train could comprise a 10 liter vessel, a 100 liter vessel, and a 1000 liter vessel). Preferably, the biomass of the recombinant microorganism is increased without the production of alcohol from fermentable carbon substrates. In the seed train, the recombinant microorganism can be supplied with excess oxygen and the carbon from the fermentable carbon substrates can be directed to respiration to increase the amplification of the biomass.

“Propagation vessel” as used herein, means the vessel in which the biomass of the recombinant microorganism is increased prior to placing in the fermentation vessel. Preferably the biomass of the recombinant microorganism is increased without the production of alcohol from sugars in the medium. The carbon from the sugars can be directed to biomass formation during the propagation phase.

“Fermentation vessel” as used herein, means the vessel in which the fermentation reaction is carried out whereby product alcohol, such as butanol, is made from a fermentable carbon substrate. “Fermentor” may be used herein interchangeably with “fermentation vessel.”

The term “recovery,” “recovering,” or variants thereof refers to removing a chemical compound from an initial mixture to obtain the compound in greater purity or at a higher concentration than the purity or concentration of the compound in the initial mixture.

“Extractant” as used herein, means a solvent used to remove or separate a product alcohol such as butanol. From time to time, as used herein the term “solvent” may be used synonymously with “extractant.” For the processes described herein, extractants are water immiscible.

“Water immiscible” or “insoluble” as used herein, refers to a chemical component such as an extractant or solvent, which is incapable of mixing with an aqueous solution such as a fermentation broth, in such a manner as to form one liquid phase.

The term “aqueous phase” as used herein, refers to the aqueous phase of a biphasic mixture obtained by contacting a fermentation broth with a water-immiscible organic extractant. In an embodiment of a process described herein that includes fermentative extraction, the term “fermentation broth” then specifically refers to the aqueous phase in biphasic fermentative extraction, and the terms “solvent-poor phase” may be used synonymously with “aqueous phase” and “fermentation broth.” In addition, undissolved solids (e.g., grain solids) can be present in the fermentation broth, such that the biphasic mixture includes the undissolved solids which are primarily dispersed in the aqueous phase.

The term “organic phase” as used herein, refers to the non-aqueous phase of a biphasic mixture obtained by contacting a fermentation broth with a water-immiscible organic extractant. From time to time, as used herein the terms “solvent-rich phase” may be used synonymously with “organic phase.”

The term “fatty acid” as used herein, refers to a carboxylic acid (e.g., aliphatic monocarboxylic acid) having C₄ to C₂₈ carbon atoms (most commonly C₁₂ to C₂₄ carbon atoms), which is either saturated or unsaturated. Fatty acids may also be branched or unbranched. Fatty acids may be derived from, or contained in esterified form, in an animal or vegetable fat, oil, or wax. Fatty acids may occur naturally in the form of glycerides in fats and fatty oils or may be obtained by hydrolysis of fats or by synthesis. The term fatty acid may describe a single chemical species or a mixture of fatty acids. In addition, the term fatty acid also encompasses free fatty acids.

The term “fatty alcohol” as used herein, refers to an alcohol having an aliphatic chain of C₄ to C₂₂ carbon atoms, which is either saturated or unsaturated.

The term “fatty aldehyde” as used herein, refers to an aldehyde having an aliphatic chain of C₄ to C₂₂ carbon atoms, which is either saturated or unsaturated.

The term “fatty amide” as used herein, refers to an amide having a long, aliphatic chain of C₄ to C₂₂ carbon atoms, which is either saturated or unsaturated.

The term “fatty ester” as used herein, refers to an ester having a long aliphatic chain of C₄ to C₂₂ carbon atoms, which is either saturated or unsaturated.

The term “carboxylic acid” as used herein, refers to any organic compound with the general chemical formula —COOH in which a carbon atom is bonded to an oxygen atom by a double bond to make a carbonyl group (—C═O) and to a hydroxyl group (—OH) by a single bond. Examples of carboxylic acids can include, but are not limited to, butyric acid, valeric acid, propanoic acid, formic acid, and acetic acid.

“Butyric acid” as used herein refers to butyric acid, isomers, and derivatives thereof, which can include isobutyric acid, hydroxyisobutyric acid, and methylbutyric acid, either individually or as mixtures thereof. From time to time, the term “butanoic acid” can be used interchangeably with “butyric acid.”

“Valerie acid” as used herein refers to valeric acid, isomers, and derivatives thereof, which can include isovaleric acid, ketoisovaleric acid, hydroxyisovaleric acid, dihydroxyisovaleric acid, methylvaleric acid, and ethylvaleric acid, either individually or as mixtures thereof. From time to time, the term “pentanoic acid” can be used interchangeably with “valeric acid.”

“Propanoic acid” as used herein refers to propanoic acid, isomers, and derivatives thereof, which can include pyruvic acid, lactic acid, methylpropanoic acid, and hydroxypropanoic acid, either individually or as mixtures thereof. From time to time, the term “propanoic acid” can be used interchangeably with “carboxyethane.”

“Acetic acid” as used herein refers to acetic acid, isomers, and derivatives thereof, which can include hydroxyacetic acid (glycolic acid), either individually or as mixtures thereof “Acetic acid” can be used interchangeably with “ethanoic acid.”

“Formic acid” can be used interchangeably with “methanoic acid.”

“Portion” as used herein, includes a part of a whole or the whole. For example, a portion of fermentation broth includes a part of the fermentation broth as well as the whole (or all) the fermentation broth.

“Partition coefficient” refers to the ratio of the concentration of a compound in the two phases of a mixture of two immiscible solvents at equilibrium. A partition coefficient is a measure of the differential solubility of a compound between two immiscible solvents. Partition coefficient, as used herein, is synonymous with the term distribution coefficient.

As used herein, the term “recombinant microorganism” refers to microorganisms such as bacteria or yeast, that are modified by use of recombinant DNA techniques, for example, by engineering a host cell to comprise a biosynthetic pathway such as a biosynthetic pathway to produce an alcohol such as butanol.

A recombinant host cell comprising an “engineered alcohol production pathway” (such as an engineered butanol or isobutanol production pathway) refers to a host cell containing a modified pathway that produces alcohol in a manner different than that normally present in the host cell. Such differences include production of an alcohol not typically produced by the host cell, or increased or more efficient production.

The term “heterologous biosynthetic pathway” as used herein refers to an enzyme pathway to produce a product in which at least one of the enzymes is not endogenous to the host cell containing the biosynthetic pathway.

The term “butanol biosynthetic pathway” as used herein refers to the enzymatic pathway to produce 1-butanol, 2-butanol, or isobutanol.

The term “1-butanol biosynthetic pathway” refers to an enzymatic pathway to produce 1-butanol. A “1-butanol biosynthetic pathway” can refer to an enzyme pathway to produce 1-butanol from acetyl-coenzyme A (acetyl-CoA). For example, 1-butanol biosynthetic pathways are disclosed in U.S. Patent Application Publication No. 2008/0182308 and International Publication No. WO 2007/041269, which are herein incorporated by reference in their entireties.

The term “2-butanol biosynthetic pathway” refers to an enzymatic pathway to produce 2-butanol. A “2-butanol biosynthetic pathway” can refer to an enzyme pathway to produce 2-butanol from pyruvate. For example, 2-butanol biosynthetic pathways are disclosed in U.S. Pat. No. 8,206,970, U.S. Patent Application Publication No. 2007/0292927, International Publication Nos. WO 2007/130518 and WO 2007/130521, which are herein incorporated by reference in their entireties.

The term “isobutanol biosynthetic pathway” refers to an enzymatic pathway to produce isobutanol. An “isobutanol biosynthetic pathway” can refer to an enzyme pathway to produce isobutanol from pyruvate. For example, isobutanol biosynthetic pathways are disclosed in U.S. Pat. No. 7,851,188, U.S. Application Publication No. 2007/0092957, and International Publication No. WO 2007/050671, which are herein incorporated by reference in their entireties. From time to time “isobutanol biosynthetic pathway” is used synonymously with “isobutanol production pathway.”

“Reducing the activity of a carboxylic acid” as used herein refers to a reduction or decrease in the acidity of the composition in which the carboxylic acid is a component. By way of an example, the activity of the carboxylic acid can be reduced by increasing the pH of the composition by addition of a base, which can neutralize the carboxylic acid to its corresponding salt. “Reducing the activity of a carboxylic acid” can also be indicated by a reduction in the level of carboxylic acid in the composition. By way of an example, the carboxylic acid can be modified to an ester by way of an esterifying agent, which would reduce the level of the carboxylic acid in the composition. By way of another example, the pH of the composition comprising the carboxylic acid can be decreased, which can increase the efficiency of carboxylic acid extraction by an extractant, thus reducing the level of the carboxylic acid in the composition. By way of another example, the composition comprising the carboxylic acid can be treated with a sequestering agent (e.g., the agent could be a weak base anion exchange resin) to sequenster the carboxylic acid (e.g., isobutyric acid) from the product alcohol (e.g., butanol).

One skilled in the art would understand that at a pH below the pKa of a carboxylic acid, the carboxylic acid can be considered predominantly in a protonated form. In the protonated form, the compound can be volatilized under certain conditions of temperature and pressure and can be absorbed preferentially into an organic liquid over an aqueous phase. One skilled in the art would understand that at a pH above the pKa of the carboxylic acid, the carboxylic acid can be considered predominantly in an unprotonated or deprotonated form. In the deprotonated form, the compound can have little or no vapor pressure and little or no solubility in an organic liquid. The dependency of the volatility and organic solubility of a carboxylic acid on pH can be used to separate it from a product alcohol.

“Increasing the pH” as used herein, refers to a process wherein an increase in the measurable pH units of the composition occurs. For example, a base is added to a composition pH 5, and the addition of the base increases the pH of the composition to pH 6. “Increasing the pH” can indicate that the resultant composition is more basic than the starting composition.

“Decreasing the pH” as used herein, refers to a process wherein a decrease in the measurable pH units of the composition occurs. For example, an acid is added to a composition pH 5, and the addition of the acid decreases the pH of the composition to pH 4. “Decreasing the pH” can indicate that the resultant composition is more acidic than the starting composition.

### Reducing Activity of Carboxylic Acids

In fermentation processes where product alcohols are produced, the fermentation processes may also result in the co-production of carboxylic acids. When the product alcohol is removed from the fermentation medium and distilled, at least a portion of these carboxylic acids can be removed and distilled such that the final purified product alcohol may have as much as 1-5% carboxylic acid content. In an ethanol production, acetic acid can be produced with high titer; however, due to the properties of ethanol and acetic acid, during the distillation and rectification process the ethanol is separated from the acetic acid and the final ethanol product has a minimum amount of acetic acid content. However, in the production of four or more carbon alcohol products, the carboxylic acid co-products can form an azeotropic mixture with the alcohol and water, not allowing for the separation of the product alcohol and carboxylic acid through distillation and further rectification. By way of an example, during the fermentive production of isobutanol, isobutyric acid may be distilled with the isobutanol at levels around 1-5% of the final product. Depending on the type of and end use of the product alcohol it may be desirable to remove any carboxylic acid co-products from the product alcohol.

Provided herein are processes for adjusting a fermentation medium to reduce the activity of one or more carboxylic acids. The processes comprise (a) providing a recombinant microorganism comprising an engineered butanol biosynthetic pathway, (b) contacting the recombinant microorganism with a fermentation medium whereby butanol is produced and wherein the fermentation medium comprises one or more carboxylic acids, and (c) adjusting the fermentation medium to reduce the activity of the one or more carboxylic acids. The process can further comprise a distillation step (d), wherein the distillation step results in the isolation of the butanol.

Also provided are processes for reducing the activity of one or more carboxylic acids in a feed. The processes comprise (a) providing a feed from a fermentation vessel, wherein the feed comprises a composition produced by a recombinant microorganism comprising an engineered butanol biosynthetic pathway, wherein the composition comprises butanol, water, and one or more carboxylic acids; and (b) adjusting the feed, wherein adjusting the feed reduces the activity of the one or more carboxylic acids. Adjusting the feed can, for example, comprise contacting the feed with an agent to reduce the activity of the one or more carboxylic acids. The processes can further comprise a distillation step (c), wherein the distillation step results in the isolation of the butanol from the composition.

Optionally, reducing the activity of the one or more carboxylic acids may include increasing the pH of the fermentation medium. Increasing the pH of the fermentation medium can, for example, result in the neutralization of the carboxylic acid, thus reducing the activity of the carboxylic acid. Increasing the pH of the fermentation medium can, for example, be accomplished by the addition of an agent to the fermentation medium (e.g., a base).

Optionally, reducing the activity of the one or more carboxylic acids may include decreasing the pH of the fermentation medium. Decreasing the pH of the fermentation medium can, for example, result in an increase in the efficiency of an extractant to extract the carboxylic acid from the fermentation medium, thus reducing the activity of the carboxylic acid in the fermentation medium. Decreasing the pH of the fermentation medium can, for example, be accomplished by the addition of an agent to the fermentation medium (e.g., an acid).

Optionally, reducing the activity of the one or more carboxylic acids include chemically modifying the one or more carboxylic acids. Chemically modifying the one or more carboxylic acids can, for example, result in the production of a desired co-product (e.g., a fragrant ester such as a butyrate). Thus, chemical modification of the carboxylic acid can result in an increase in the total product produced by the fermentation process. Without intending to be limited by theory, the carboxylic acids can be chemically modified by the addition of an agent to the fermentation medium. The agent can be an esterifying agent (e.g., a lipase). The esterifying agent can be used in conjunction with the product alcohol to esterify the carboxylic acid. Alternatively, the product alcohol (e.g., isobutanol) or an exogenous alcohol (e.g., methanol) can be used to esterify the carboxylic acid (e.g., isobutyric acid). Proper agents and methods to chemically modify a carboxylic acid are known by those skilled in the art, see, e.g., Ikeda et al., J. Chem. Technol. Biotechnol. 77:86-91 (2001); Pereira et al., Appl. Biochem. Biotechnol. 98-100:977-86 (2002); U.S. Patent Application No. 2010/0124773; U.S. Patent Application No. 2011/0312044; U.S. Patent Application No. 2013/0071891; U.S. Pat. No. 5,780,275; and U.S. Pat. No. 8,409,834. Agents for enabling an esterification reaction can include, but are not limited to, sulfuric acid and acidic ion exchange resins.

Optionally, reducing the activity of the one or more carboxylic acids includes sequestering the one or more carboxylic acids from the product butanol. Sequestering the one or more carboxylic acids can, for example, result in the improved isolation of the product alcohol (e.g., butanol). By way of an example, a strong base membrane, molecular sieve, anion exchange resin, or phase transfer catalyst could be used to sequester the carboxylic acids such that the carboxylic acids are removed from the final alcohol product. Electrodialysis can be used for membrane separation of an unprotonated carboxylic acid (e.g., an unprotonated isobutyric acid), such that the product alcohol (e.g., isobutanol) is on one side of the membrane and the carboxylic acid is on the other side of the membrane, thus allowing for the isolation of the product alcohol. By way of another example, the carboxylic acids can be sequestered with a sequestering agent that binds or associates with the carboxylic acid and prevents the carboxylic acid from being distilled with the butanol. Sequestering agents are known in the art. Examples of anion exchange resin can include, but are not limited to, polyethyleneimine (PEI), DOWEX® TAN-1 (Dow Chemical Company, Midland, Mich.), Diaion® WA30 (SUPELCO, Sigma Aldrich; St. Louis, Mo.), Amberlite® IRA-67 (FLUKA, Sigma Aldrich), Amberlite® IRA-96 (FLUKA, Sigma Aldrich), and polyAPTAC. An example of a strong base molecular sieve can include, but is not limited to, zeolite treated with magnesium oxide. An example of a phase transfer catalyst can include, but is not limited to, long chain alkyl trimethyl ammonium chloride salt, which can be used in conjunction with a liquid-liquid extraction ISPR process.

Adjusting the fermentation medium can, for example, include contacting the fermentation medium with an agent. The contacting step can, for example, occur during the seed train and/or propagation phases of the fermentation process for the recombinant microorganism. By way of an example, the fermentation medium can be contacted with an agent (e.g., a base) to increase the pH of the fermentation medium during the seed train and/or propagation phases. Optionally, the contacting step can occur during the production phase of the fermentation. By way of an example, the fermentation medium can be contacted with an agent (e.g., a base or an acid) to increase or decrease the pH depending on the desired route of reducing the activity of the one or more carboxylic acids. To reduce the activity of the carboxylic acid by neutralizing the carboxylic acid, a base can be added to the fermentation medium, increasing the pH of the fermentation medium, whereby the one or more carboxylic acids are neutralized to the one or more corresponding salts. To reduce the activity of the carboxylic acid by decreasing the level of the carboxylic acid in the fermentation medium, an acid can be added to the fermentation medium, decreasing the pH of the fermentation medium, whereby addition of an extractant leads to the efficient extraction of the one or more carboxylic acids from the fermentation medium and a reduction in the level of the carboxylic acid in the fermentation medium. By way of another example, the fermentation medium can be contacted with an esterifying agent, which can esterify the one or more carboxylic acids, reducing the level of the one or more carboxylic acids in the fermentation medium and, thus reducing the activity of the one or more carboxylic acids. Reducing the activity of the one or more carboxylic acids in the fermentation medium can lead to beneficial effects for the fermentation. These beneficial effects can include, but are not limited to, an increase in growth rate of the microorganism, an increase in production of alcohol (e.g., isobutanol) from the microorganism, an increase in biomass production of the microorganism, and an increase in the fermentable carbon substrate (e.g., glucose) consumption by the microorganism.

Optionally, adjusting the fermentation medium further comprises distilling the fermentation medium, whereby the distillation step results in the isolation of the butanol from the composition. Optionally, the contacting step occurs after fermentation and prior to distillation. By way of an example, the contacting step can occur in one or more beer wells prior to distillation. By way of another example, the contacting step can occur in one or more external extractor units (e.g., a siphon, a decanter, a centrifuge, a gravity settler, a phase splitter, a mixer-settler, a column extractor, a centrifugal extractor, a hydrocyclone spray tower, or combinations thereof) prior to distillation. Examples of external extractor units are described in U.S. application Ser. No. 13/828,353, which is herein incorporated by reference in its entirety. Optionally, the contacting step occurs during distillation. Optionally, the contacting step occurs during fermentation, prior to distillation, and/or during distillation. The distillation step can, for example, comprise a distillation system comprising at least one distillation column and at least one decanter vessel. By way of an example, the contacting step during distillation can occur in the at least one decanter vessel.

Optionally, the adjusting step can occur after the distillation step. The adjusting step can occur in a recycle stream from the distillation system. By way of an example, the recycle stream can be provided to an anaerobic digester, which contains an agent (i.e., a microorganism) capable of degrading or destroying the carboxylic acids in the recycle stream.

Optionally, the agent can be selected from the group consisting of a sequestering agent, a complexation agent, a neutralizing agent, a modifying agent, a phase-changing agent, and a destructive agent.

Optionally, the agent can be a neutralizing agent. A neutralizing agent can, for example, be an agent that neutralizes the acidity of the carboxylic acid, for example by increasing the pH of the composition in which the carboxylic acid is present. Optionally, the neutralizing agent is a base. The base can be selected from, but is not limited to, the group consisting of calcium oxide, calcium hydroxide, calcium carbonate, sodium hydroxide, sodium carbonate, sodium phosphate, sodium ethoxide, potassium hydroxide, potassium carbonate, potassium phosphate, magnesium hydroxide, ammonium hydroxide, barium hydroxide, aluminum hydroxide, ferrous hydroxide, ferric hydroxide, zinc hydroxide, lithium hydroxide, and combinations thereof. Optionally, the neutralizing agent can be selected from, but is not limited to, the group consisting of urea, fatty amines, anhydrous ammonia, and ion exchange resin.

By way of an example, neutralizing agents can be added to the fermentation medium comprising butanol and one or more carboxylic acids in the seed train, propagation vessel, and/or the fermentation vessel. For example, a base, such as ammonium hydroxide, calcium hydroxide, ammonium sulfate, calcium carbonate, sodium carbonate, and/or potassium carbonate can be added to the fermentation medium, thus, resulting in an increase in pH of the fermentation medium and a reduction in activity of the carboxylic acid, as the carboxylic acid is neutralized to its corresponding salt. Without intending to be limited by theory, in the fermentation of isobutanol, the activity of the co-produced isobutyric acid can be reduced by the addition of calcium carbonate to the fermentation medium, which results in the formation of the corresponding butyric salt and reduction in activity of isobutyric acid. Those skilled in the art will understand which neutralizing agent and the amount of neutralizing agent to achieve the greatest reduction in carboxylic activity while maintaining the highest level of growth and production in the fermentation process. Optionally, the choice of neutralizing agent to add to the fermentation medium can result in additional benefits for the fermentation process. The additional benefits due to the neutralizing agent can include, but are not limited to, an increase in growth rate of the microorganism, an increase in production of isobutanol from the microorganism, an increase in biomass production of the microorganism, and an increase in the fermentable sugar (e.g., glucose) consumption by the microorganism.

A person skilled in the art would want to increase the pH of the fermentation medium above the pKa of the carboxylic acid (e.g., the pKa of isobutyric acid is approximately 4.86) to neutralize the carboxylic to its corresponding salt. Optionally, the pH can be increased at least one pH unit above the pKa of the carboxylic acid. Optionally, the pH can be increased two, three, four, five, or six pH units above the pKa of the carboxylic acid. The pH can also be increased in non-integer values above the pKa of the carboxylic acid. An increase in the pH of the fermentation medium can lead to other beneficial effects for the fermentation process. These beneficial effects can include, but are not limited to, an increase in growth rate of the microorganism, an increase in production of isobutanol from the microorganism, an increase in biomass production of the microorganism, and an increase in the fermentable sugar (e.g., glucose) consumption by the microorganism.

Optionally, the agent can be a modifying agent. Without intending to be limited by theory, a modifying agent can, for example, be an agent that changes the chemistry of the carboxylic acid such that the carboxylic acid is not capable of being co-purified with the product alcohol. Optionally, the carboxylic acid chemistry is altered such that the co-purification with the product alcohol will not affect the desired end use of the product alcohol. Optionally, the carboxylic acid chemistry is altered to form a desired co-product. The modifying agent can, for example, be an esterifying agent. By way of an example, an esterifying agent can convert the isobutyric acid produced during an isobutanol fermentation into an ester (i.e., isobutyl isobutyrate) that can be co-purified with isobutanol and used in the desired end use of the isobutanol (e.g., as a fuel) or purified separately as a co-product (e.g., as a fragrance chemical). In certain embodiments, the modifying agent can include oxidizing agents. An oxidizing agent can include, but is not limited to, ferric salts, thallic salts, potassium permanganate, potassium dichromate, peroxides, percarbonates or persulfates that may have the effect of transforming the carboxylic acid into other compounds that are more easily separated from the product alcohol. For example, potassium permanganate can under certain conditions oxidize butyric acid to CO₂.

By way of another example, a modifying agent (e.g., an agent that catalyzes an oxidation step) can be used to convert isobutyric acid to 2-hydroxy-isobutyric acid. See, e.g., Beckwith et al., Australian J. Chem. 18(7):1023-33 (1965) and U.S. Pat. Nos. 4,448,985 and 4,450,293. Conversion of isobutyric acid to 2-hydroxy-isobutyric acid in the presence of calcium ions can form a calcium ion chelate, which can subsequently be precipitated and separated from isobutanol. See, e.g., Johnston et al., New Zealand J. Sci. Technol. 37B:522-37 (Section A) (1956); Piispanen et al., Acta Chemica Scandinavica 49(4):235-40 (1995).

By way of another example, a modifying agent can be used to convert isobutyric acid to isobutanol by way of hydrogenation of the isobutyric acid. The agent can be a transition-metal catalyst. The transition-metal catalyst can be used in an aqueous solution comprising butanol. See, e.g., Lee et al., Bulletin of the Korean Chemical Society 28(11):2034-40 (2007); Lee et al., Industrial Eng. Chem. 13(7):1067-75 (2007); Mao et al., Polymers Advanced Technol. 14(3-5):278-81 (2003); and Chen et al., J. Mol. Catalysis. A:Chemical 351:217-27 (2011). In certain embodiments, reduction of isobutyric acid may be accomplished with the use of reducing agents that can include, but is not limited to, lithium aluminum hydride, sodium borohydride, sulfite salts, phosphine compounds, and hydroxylamine.

Optionally, the agent can be a complexation agent. Without intending to be limited by theory, a complexation agent can, for example, be an agent that forms a complex with the carboxylic acid and prevents the carboxylic acid from being co-purified with the alcohol product. By way of an example, complexation agents can be used to complex a deprotonated form of the carboxylic acid (e.g., the deprotonated from of isobutyric acid), such that the carboxylic acid is not extracted with the product alcohol during the extraction phase. Optionally, the complexed carboxylic acid can be extracted with an extractant (e.g., oleyl alcohol) that prefers the complexed carboxylic acid, which can allow for the extraction of the product alcohol with a separate extractant. Examples of complexation agents can include, but are not limited to, metal ions such as Ca²⁺, Mg²⁺, Fe³⁺, and Ti⁴⁺. In certain embodiments, tetrabutyltitanate can be added to form the complex titanium tetrabutyrate.

Optionally, the agent can be a sequestering agent. A sequestering agent can, for example, result in the improved isolation of the product alcohol (e.g., butanol) by sequestering the carboxylic acid such that the carboxylic acid is not co-purified with the alcohol product. A sequestering agent can bind or associate with the carboxylic acid and prevent the carboxylic acid from being distilled with the butanol. By way of an example, the carboxylic acids can be sequestered in the organic phase by using an extractant (e.g., fatty acid butyl esters (FABE)) with a low affinity for the carboxylic acid (e.g., isobutyric acid). Thus, the product alcohol (e.g., isobutanol) can be extracted and subsequently isolated with little to no carboxylic acids (e.g., isobutyric acid). By way of another example, the sequestering agents can be anion exchange resins that bind and sequester the carboxylic acids in the composition to allow for the product alcohol (e.g., isobutanol) to be extracted and subsequently isolated with little to no carboxylic acids (e.g., isobutyric acid). Sequestering agents are known in the art.

In certain embodiments, liquid-liquid extraction is employed to remove the product alcohol (e.g., butanol) from the fermentation medium. In certain extractants, carboxylic acids (e.g., isobutyric acid) can also be removed from fermentation medium. To enhance the removal of the one or more carboxylic acids from the fermentation medium, a sequestering agent such as a phase transfer agent can be added to the extractant to form a solvent mixture. The phase transfer agent can provide chemisorption to the one or more carboxylic acids present in the fermentation medium. To one skilled in the art, chemisorption is a form of absorption where a chemical bond is formed between the solute and the absorbing solvent. A phase transfer agent can include, but is not limited to, hexadecyl trimethyl ammonium chloride. The chemically absorbed one or more carboxylic acids can be later released from the extractant solvent mixture before recycling it to the fermentation medium in a multi-pass extraction configuration.

Optionally, in embodiments where liquid-liquid extraction is employed to remove the product alcohol (e.g., butanol) from the fermentation medium, the pH of the fermentation medium can be decreased to increase the efficiency of the extractant to extract one or more carboxylic acids from the fermentation medium. The pH of the fermentation medium can be decreased by addition of an agent (e.g., an acid). The acid can be selected from, but is not limited to, the group consisting of hydrochloric acid, hydrofluoric acid, hydrobromic acid, hydroiodic acid, hydrosulfuric acid, sulfuric acid, nitric acid, nitrous acid, hypochlorous acid, chlorous acid, chloric acid, perchloric acid, sulfurous acid, phosphoric acid, phosphorous acid, boric acid, silicic acid, carbonic acid, acetic acid, oxalic acid, uric acid, lactic acid, citric acid, and combinations thereof. Optionally, the choice of acid to add to the fermentation medium can result in additional benefits for the fermentation process. The additional benefits can include, but are not limited to, an increase in growth rate of the microorganism, an increase in production of isobutanol from the microorganism, an increase in biomass production of the microorganism, and an increase in the fermentable sugar (e.g., glucose) consumption by the microorganism.

One of skill in the art would understand that a decrease in the pH of the fermentation medium below the pKa of the carboxylic acid (e.g., the pKa of isobutyric acid is approximately 4.86) would allow for the carboxylic acid to be predominantly in its protonated form. Optionally, the pH can be decreased at least one unit below the pKa of the carboxylic acid. Optionally, the pH can be decreased two, three, four, five, or six pH units below the pKa of the carboxylic acid. The pH can also be decreased in non-integer values below the pKa of the carboxylic acid. A person skilled in the art would readily be able to determine the desired pH for each contemplated extractant to efficiently extract the one or more carboxylic acids.

Optionally, the agent can be a destructive agent. A destructive agent can, for example, destroy the carboxylic acid such that the carboxylic acid is no longer capable of being distilled with the butanol product. Optionally, a destructive agent can destroy the carboxylic acid in a recycle stream from the distillation system such that the carboxylic acid is not recycled to the front end of the fermentation process. Without intending to be limited by theory, a destructive agent can specifically target and destroy the intended carboxylic acid (e.g., in an isobutanol fermentation, the destructive agent can destroy isobutyric acid). By way of an example, a destructive agent can be a microorganism that specifically targets carboxylic acids (e.g., isobutyric acid) to remove the compound from the aqueous solution. Preferably the microorganism can produce a useful product from the destruction of the carboxylic acid. The microorganism can be a recombinant microorganism designed to target carboxylic acids. The microorganism can be present in an anaerobic digestor. The microorganism can biologically degrade the carboxylic acids in a stream provided to the anaerobic digester to form methane and carbon dioxide. A destructive agent can, for example, oxidatively degrade the carboxylic acid

(e.g., isobutyric acid) into more volatile compounds such as CO₂ and acetone. Optionally, a destructive agent can oxidize isobutyric acid to hydroisobutyric acid, which is less volatile and does not form an azeotropic mixture with water.

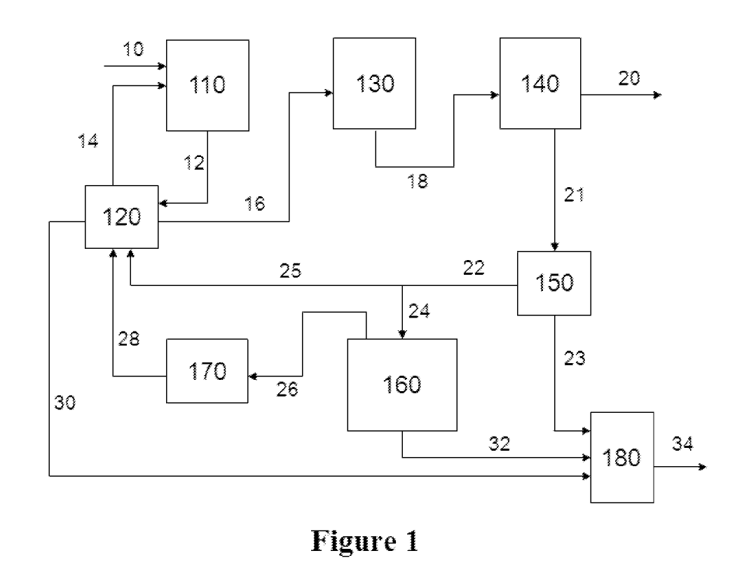

FIG. 1 depicts a process flow diagram to manufacture a product alcohol (e.g., isobutanol). Dry ground corn stream 10 may be added to a cooking step 110 along with recycled water stream 14 to form a liquefied mash stream 12. The suspended grain solids present in stream 12 may be largely removed in solids separation step 120 to form a thin mash stream 16. The separated solids may be washed repeatedly using the recycled water streams 25 and 28 in order to recover most of the fermentable compounds from the final washed wet cake stream 30. Either of streams 25 or 28 may contain some level of recycled carboxylic acids in both protonated and deprotonated forms. In certain embodiments, an agent may be added to treat one or more of the aqueous streams internal to step 120 that can result in the precipitation of the carboxylic acids and these insoluble salts that result may be removed with the wet cake stream 30. For example, a heterogeneous sequestering agent such as an anionic exchange resin in the form of beads may be used to chemisorb the carboxylic acids (e.g., isobutyric acid) from the aqueous phase of a treated stream. The resin can then be contained with the grain solids to form a separable wet cake. Thin mash stream 16 may be forwarded to the propagation and production phases 130 where microorganisms can convert fermentable compounds into the product alcohol (e.g., isobutanol) that will be contained in aqueous beer stream 18. Any carboxylic acid that may form in this step can be treated using any of the agents described herein. Stream 18 can then be directed to distillation step 140 where purified product alcohol stream 20 is produced. The stillage stream 21 resulting from the removal of purified product alcohol may contain carboxylic acids and these can be treated here. For example, a sequestering agent such as anionic exchange resin in the form of beads may be added to stream 21 prior to entering solids separation area 150 and the beads that may chemisorb at least a portion of the carboxylic acids (e.g., isobutyric acid) will be contained with grain solids to form a separable wet cake stream 23. A portion (stream 25) of the thin stillage stream 22 may form backset for recycle and another portion, stream 24, may form feed to evaporation area 160. The water vapor and condensate stream 26 produced by evaporation area 160 may contain at least some of the carboxylic acids that were present in stream 24. At least a portion of these carboxylic acids contained in stream 26 may be degraded biologically in anaerobic digester 170 to form methane and CO₂ such that the carboxylic acids content of recycle water stream 28 is reduced. A concentrated syrup stream 32 that is formed from evaporation may be combined with wet cakes 30 and 23 in a drying area 180 to form a DDGS co-product stream 34.

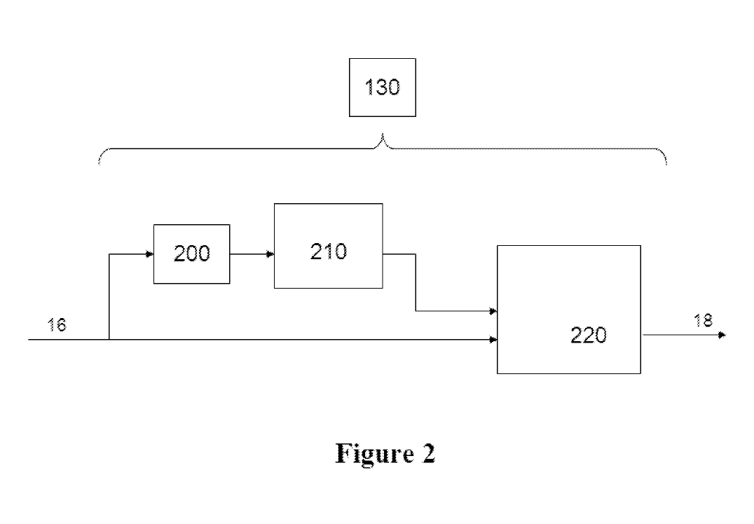

FIG. 2 illustrates a typical configuration of the propagation and fermentation area 130. A portion of mash stream 16 may be diverted to a seed area 200 and subsequently forwarded to a propagation area 210 where aerobic conditions are maintained using air. Under these conditions, some carboxylic acids may form and may inhibit the growth rate of the microorganisms. Any of the agents described herein may be used to maintain low concentration of carboxylic acids in areas 200 and 210. For example, an agent (e.g., a base) can be added to the seed and propagation media to increase the pH of the media, resulting in the neutralization of the carboxylic acids. By way of another example, a heterogeneous sequestering agent such as an anionic exchange resin in the form of beads may be used to chemisorb the carboxylic acids (e.g., isobutyric acid) from the aqueous phase of a propagation vessel and these beads can be screened from the broth during discharge. Furthermore, under certain conditions, an oxidizing agent may be used to remove carboxylic acids during an aerobic growth phase. The discharge of propagation area 210 containing microorganisms is combined with a portion of mash stream 16 in fermentation area 220 to from beer stream 18.

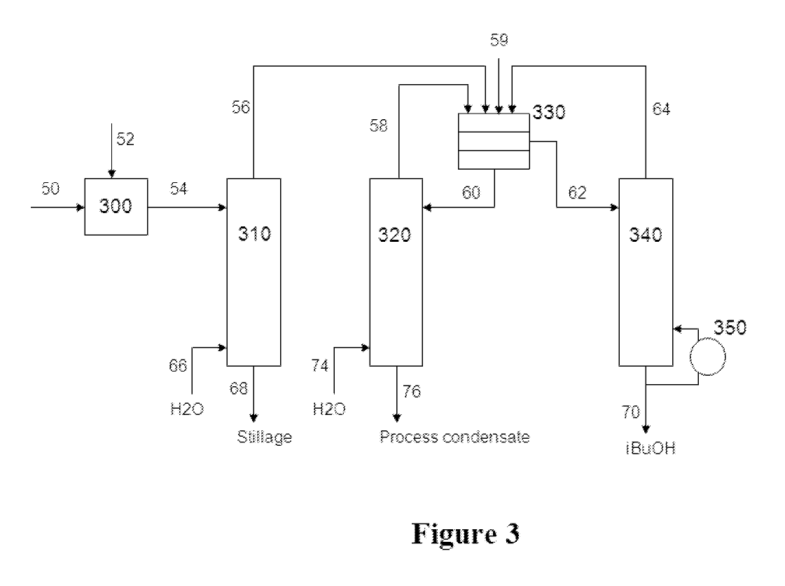

A typical distillation configuration for purifying a product alcohol (e.g., isobutanol) is provided in FIG. 3. Stream 50 comprising the product alcohol (e.g., isobutanol) may be combined with a treating agent 52 in treatment area 300 such that no carboxylic acid content is present in stream 54. By way of an example, the treating agent 52 can increase the pH of stream 50, thus neutralizing the carboxylic acids. The treatment area 300 can, for example, be one or more beer wells or one or more mixer-settlers. Optionally, the treating agent 52 can be combined with stream 50 in treatment area 300, which can result in a stream 54 with carboxylic acids that can be separated from the product alcohol (e.g., isobutanol) in stripping column 310. For example, stream 52 may comprise NH₃ that reacts with the isobutyric acid to form ammonium isobutyrate. The overhead vapor stream 56 may contain isobutanol and water and very little isobutyric acid. Water vapor stream 66 is injected into the bottom of stripping column 310 such that very little isobutanol remains in the bottoms stillage stream 68. Overhead vapor stream 56 is condensed and decanted into phase separated aqueous and organic layers in vessel 330. A treating agent may be added via stream 59 directly into decanter vessel 330 to transfer any carboxylic acids entering the vessel away from the organic layer and into the aqueous layer. Some organic compounds that azeotrope with isobutyric acid, for example, but not with isobutanol may also be added into the decanter vessel. Examples of these compounds include hydrocarbons such as alkanes like hexane or heptane and these will reside predominantly in the organic layer. When organic outlet stream 62 from the decanter vessel 330 is fed to a rectifier column 340 equipped with reboiler 350, the isobutanol becomes purified out of the bottom of the column in stream 70 and the vapor overhead stream 64 may be condensed back into decanter vessel 330. In some embodiments where carboxylic acid compounds are contained in organic stream 62, an alternative product stream may be drawn from the side of rectifier column 340 at a tray location where the carboxylic acid concentration is low. To one skilled in the art, the low moisture conditions of the interior of rectifier column 340 are favorable for separating isobutanol from isobutyric acid. The aqueous outlet stream 60 is stripped in column 320 using water vapor stream 74 to produce bottoms stream 76 and to recover isobutanol into vapor overhead stream 58 that may be condensed into the decanter vessel 330.

One of skill in the art will recognize that reducing the activity of the one or more carboxylic acids can occur in one or more areas of the alcohol production process. A non-limiting example can include a first adjustment to the fermentation medium, which can be made during the fermentation process (e.g., an agent can be added to the seed train, propagation vessel, and/or fermentation vessel), and a second adjustment to the composition, which can be made post fermentation prior to distillation (e.g., an agent can be added to the beer well). By way of another non-limiting example, a first adjustment can be made post fermentation prior to distillation and a second adjustment can be made during distillation (e.g., an agent can be added to the decanter during distillation). By way of another example, a first adjustment can be made in the fermentation process, a second adjustment can be made post fermentation pre-distillation, and a third adjustment can be made during distillation. By way of another example, a first adjustment can be made in the fermentation process, a second adjustment can be made post fermentation-pre distillation, a third adjustment can be made during distillation, and a fourth adjustment can be made post-distillation in a recycle stream.

Also provided herein are compositions produced by the processes described herein, wherein the composition comprises less than 1 weight percent carboxylic acid. The composition can comprise less than 0.10 weight percent carboxylic acid, preferably less than 0.01 weight percent carboxylic acid, and most preferably less than 0.001 weight percent carboxylic acid.

Identities and levels of the one or more carboxylic acids in a butanol based composition can, for example, be determined using methods selected from, but not limited to, gas chromatography (GC), gas chromatography-mass spectroscopy (GC-MS), mass spectroscopy (MS), high performance liquid chromatography (HPLC), nuclear magnetic resonance (NMR) spectroscopy, near infrared (NIR) spectroscopy, and by standard titration methods. These methods are known in the art. Briefly, by way of an example, the identity and level of the one or more carboxylic acids in a butanol based composition can be determined using gas chromatography. The butanol based composition can be compared to an internal standard using a gas chromatograph, which utilizes a capillary column and a flame ionization detector (FID) under temperature programmed conditions. To detect a reduction in the level of the carboxylic acid, a butanol based composition sample can be tested prior to treatment with an agent and after treatment with the agent. A loss in the level of the specified carboxylic acid indicates a reduction in the activity (i.e., the level) of the carboxylic acid.

### Recombinant Microorganisms

While not wishing to be bound by theory, it is believed that the processes described herein are useful in conjunction with any alcohol producing microorganism, particularly recombinant microorganisms which produce alcohol.

Recombinant microorganisms which produce alcohol are also known in the art (e.g., Ohta et al., Appl. Environ. Microbiol. 57:893-900 (1991); Underwood et al., Appl. Envrion. Microbiol. 68:1071-81 (2002); Shen and Liao, Metab. Eng. 10:312-20 (2008); Hahnai et al., Appl. Environ. 73:7814-8 (2007); U.S. Pat. No. 5,514,583; U.S. Pat. No. 5,712,133; International Publication No. WO 1995/028476; Feldmann et al., Appl. Microbiol. Biotechnol. 38:354-61 (1992); Zhang et al., Science 267:240-3 (1995); U.S. Patent Publication No. 2007/0031918A1; U.S. Pat. No. 7,223,575; U.S. Pat. No. 7,741,119; U.S. Patent Publication No. 2009/0203099A1; U.S. Patent Publication No. 2009/0246846A1; and International Publication No. WO 2010/075241, which are herein incorporated by reference).

For example, the metabolic pathways of microorganisms may be genetically modified to produce butanol. These pathways may also be modified to reduce or eliminate undesired metabolites, and thereby improve yield of the product alcohol. Optionally, the microorganisms may also be modified to reduce or eliminate undesired byproducts (e.g., isobutyric acid) which may codistill with the butanol after production by the microorganism. The production of butanol by a microorganism is disclosed, for example, in U.S. Pat. Nos. 7,851,188; 7,993,889; 8,178,328, 8,206,970; U.S. Patent Application Publication Nos. 2007/0292927; 2008/0182308; 2008/0274525; 2009/0305363; 2009/0305370; 2011/0250610; 2011/0313206; 2011/0111472; 2012/0258873; and 2013/0071898, the entire contents of each are herein incorporated by reference. In some embodiments, microorganisms comprise a butanol biosynthetic pathway or a biosynthetic pathway for a butanol isomer such as 1-butanol, 2-butanol, or isobutanol. In some embodiments, the biosynthetic pathway converts pyruvate to a fermentative product. In some embodiments, the biosynthetic pathway converts pyruvate as well as amino acids to a fermentative product. In certain embodiments, at least one, at least two, at least three, at least four, or at least five polypeptides catalyzing substrate to product conversions in the butanol biosynthetic pathway are encoded by heterologous polynucleotides in the microorganism. In certain embodiments, all the polypeptides catalyzing substrate to product conversions of the butanol biosynthetic pathway are encoded by heterologous polynucleotides in the microorganism. In will be appreciated that microorganisms comprising a butanol biosynthetic pathway may further comprise one or more additional genetic modifications as disclosed in U.S. Patent Application Publication No. 2013/0071898, which is herein incorporated by reference in its entirety.

In some embodiments, the microorganism may be bacteria, cyanobacteria, filamentous fungi, or yeasts. Suitable microorganisms capable of producing product alcohol (e.g., butanol) via a biosynthetic pathway include a member of the genera Clostridium, Zymomonas, Escherichia, Salmonella, Serratia, Erwinia, Klebsiella, Shigella, Rhodococcus, Pseudomonas, Bacillus, Lactobacillus, Enterococcus, Alcaligenes, Paenibacillus, Arthrobacter, Corynebacterium, Brevibacterium, Schizosaccharomyces, Kluveromyces, Yarrowia, Pichia, Zygosaccharomyces, Debaryomyces, Candida, Brettanomyces, Pachysolen, Hansenula, Issatchenkia, Trichosporon, Yamadazyma, or Saccharomyces. In one embodiment, recombinant microorganisms may be selected from the group consisting of Escherichia coli, Alcaligenes eutrophus, Bacillus lichenifonnis, Paenibacillus macerans, Rhodocuccus erythropolis, Pseudomonas putida, Lactobacillus plantarum, Enterococcus faecium, Enterococcus gallinarium, Enterococcus faecalis, Bacillus subtilis, Candida sonorensis, Candida methanosorbosa, Kluyveromyces lactis, Kluyveromyces marxianus, Kluveromyces thermotolerans, Issatchenkia orientalis, Debaryomyces hansenii, and Saccharomyces cerevisiae. In one embodiment, the genetically modified microorganism is yeast. In one embodiment, the genetically modified microorganism is a crabtree-positive yeast selected from Saccharomyces, Zygosaccharomyces, Schizosaccharomyces, Dekkera, Torulopsis, Brettanomyces, and some species of Candida. Species of crabtree-positive yeast include, but are not limited to, Saccharomyces cerevisiae, Saccharomyces kluyveri, Schizosaccharomyces pombe, Saccharomyces bayanus Saccharomyces mikitae, Saccharomyces paradoxus, Saccharomyces uvarum, Saccharomyces castelli, Zygosaccharomyces rouxii, Zygosaccharomyces bailli, and Candida glabrata.

In some embodiments, the host cell is Saccharomyces cerevisiae. Saccharomyces cerevisiae are known in the art and are available from a variety of sources including, but not limited to, American Type Culture Collection (Rockville, Md.), Centraalbureau voor Schimmelcultures (CBS) Fungal Biodiversity Centre, LeSaffre, Gert Strand AB, Ferm Solutions, North American Bioproducts, Martrex, and Lallemand. S. cerevisiae include, but are not limited to, BY4741, CEN.PK 113-7D, Ethanol Red® yeast, Ferm Pro™ yeast, Bio-Ferm® XR yeast, Gert Strand Prestige Batch Turbo alcohol yeast, Gert Strand Pot Distillers yeast, Gert Strand Distillers Turbo yeast, FerMax™ Green yeast, FerMax™ Gold yeast, Thermosacc® yeast, BG-1, PE-2, CAT-1, CBS7959, CBS7960, and CBS7961.

In some embodiments, the microorganism may be immobilized or encapsulated. For example, the microorganism may be immobilized or encapsulated using alginate, calcium alginate, or polyacrylamide gels, or through the induction of biofilm formation onto a variety of high surface area support matrices such as diatomite, celite, diatomaceous earth, silica gels, plastics, or resins. In some embodiments, ISPR may be used in combination with immobilized or encapsulated microorganisms. This combination may improve productivity such as specific volumetric productivity, metabolic rate, product alcohol yields, and tolerance to product alcohol. In addition, immobilization and encapsulation may minimize the effects of the process conditions such as shearing on the microorganisms.

Biosynthetic pathways for the production of isobutanol that may be used include those as described by Donaldson et al. in U.S. Pat. No. 7,851,188; U.S. Pat. No. 7,993,388; and International Publication No. WO 2007/050671, which are incorporated herein by reference. In one embodiment, the isobutanol biosynthetic pathway comprises the following substrate to product conversions:

a) pyruvate to acetolactate, which may be catalyzed, for example, by acetolactate synthase;

b) the acetolactate from step a) to 2,3-dihydroxyisovalerate, which may be catalyzed, for example, by acetohydroxy acid reductoisomerase;

c) the 2,3-dihydroxyisovalerate from step b) to α-ketoisovalerate, which may be catalyzed, for example, by dihydroxyacid dehydratase;

d) the α-ketoisovalerate from step c) to isobutyraldehyde, which may be catalyzed, for example, by a branched-chain α-keto acid decarboxylase; and,

e) the isobutyraldehyde from step d) to isobutanol, which may be catalyzed, for example, by a branched-chain alcohol dehydrogenase.

In another embodiment, the isobutanol biosynthetic pathway comprises the following substrate to product conversions:

a) pyruvate to acetolactate, which may be catalyzed, for example, by acetolactate synthase;

b) the acetolactate from step a) to 2,3-dihydroxyisovalerate, which may be catalyzed, for example, by ketol-acid reductoisomerase;

c) the 2,3-dihydroxyisovalerate from step b) to a-ketoisovalerate, which may be catalyzed, for example, by dihydroxyacid dehydratase;

d) the α-ketoisovalerate from step c) to valine, which may be catalyzed, for example, by transaminase or valine dehydrogenase;

e) the valine from step d) to isobutylamine, which may be catalyzed, for example, by valine decarboxylase;

f) the isobutylamine from step e) to isobutyraldehyde, which may be catalyzed by, for example, omega transaminase; and,

g) the isobutyraldehyde from step f) to isobutanol, which may be catalyzed, for example, by a branched-chain alcohol dehydrogenase.

In another embodiment, the isobutanol biosynthetic pathway comprises the following substrate to product conversions:

a) pyruvate to acetolactate, which may be catalyzed, for example, by acetolactate synthase;

b) the acetolactate from step a) to 2,3-dihydroxyisovalerate, which may be catalyzed, for example, by acetohydroxy acid reductoisomerase;

c) the 2,3-dihydroxyisovalerate from step b) to a-ketoisovalerate, which may be catalyzed, for example, by acetohydroxy acid dehydratase;

d) the α-ketoisovalerate from step c) to isobutyryl-CoA, which may be catalyzed, for example, by branched-chain keto acid dehydrogenase;

e) the isobutyryl-CoA from step d) to isobutyraldehyde, which may be catalyzed, for example, by acylating aldehyde dehydrogenase; and,

f) the isobutyraldehyde from step e) to isobutanol, which may be catalyzed, for example, by a branched-chain alcohol dehydrogenase.

Biosynthetic pathways for the production of 1-butanol that may be used include those described in U.S. Patent Application Publication No. 2008/0182308 and WO2007/041269, which are incorporated herein by reference. In one embodiment, the 1-butanol biosynthetic pathway comprises the following substrate to product conversions:

a) acetyl-CoA to acetoacetyl-CoA, which may be catalyzed, for example, by acetyl-CoA acetyltransferase;

b) the acetoacetyl-CoA from step a) to 3-hydroxybutyryl-CoA, which may be catalyzed, for example, by 3-hydroxybutyryl-CoA dehydrogenase;

c) the 3-hydroxybutyryl-CoA from step b) to crotonyl-CoA, which may be catalyzed, for example, by crotonase;

d) the crotonyl-CoA from step c) to butyryl-CoA, which may be catalyzed, for example, by butyryl-CoA dehydrogenase;

e) the butyryl-CoA from step d) to butyraldehyde, which may be catalyzed, for example, by butyraldehyde dehydrogenase; and,

f) the butyraldehyde from step e) to 1-butanol, which may be catalyzed, for example, by butanol dehydrogenase.

Biosynthetic pathways for the production of 2-butanol that may be used include those described by Donaldson et al. in U.S. Pat. No. 8,206,970; U.S. Patent Application Publication Nos. 2007/0292927 and 2009/0155870; International Publication Nos. WO 2007/130518 and WO 2007/130521, all of which are incorporated herein by reference. In one embodiment, the 2-butanol biosynthetic pathway comprises the following substrate to product conversions:

a) pyruvate to alpha-acetolactate, which may be catalyzed, for example, by acetolactate synthase;

b) the alpha-acetolactate from step a) to acetoin, which may be catalyzed, for example, by acetolactate decarboxylase;

c) the acetoin from step b) to 3-amino-2-butanol, which may be catalyzed, for example, acetonin aminase;

d) the 3-amino-2-butanol from step c) to 3-amino-2-butanol phosphate, which may be catalyzed, for example, by aminobutanol kinase;

e) the 3-amino-2-butanol phosphate from step d) to 2-butanone, which may be catalyzed, for example, by aminobutanol phosphate phosphorylase; and,

f) the 2-butanone from step e) to 2-butanol, which may be catalyzed, for example, by butanol dehydrogenase.

In another embodiment, the 2-butanol biosynthetic pathway comprises the following substrate to product conversions:

a) pyruvate to alpha-acetolactate, which may be catalyzed, for example, by acetolactate synthase;

b) the alpha-acetolactate from step a) to acetoin, which may be catalyzed, for example, by acetolactate decarboxylase;

c) the acetoin to 2,3-butanediol from step b), which may be catalyzed, for example, by butanediol dehydrogenase;

d) the 2,3-butanediol from step c) to 2-butanone, which may be catalyzed, for example, by dial dehydratase; and,

e) the 2-butanone from step d) to 2-butanol, which may be catalyzed, for example, by butanol dehydrogenase.

### Growth for Production

Recombinant host cells disclosed herein are grown in fermentation media which contains suitable carbon substrates. Additional carbon substrates may include, but are not limited to, monosaccharides such as fructose, oligosaccharides such as lactose, maltose, galactose, or sucrose, polysaccharides such as starch or cellulose or mixtures thereof and unpurified mixtures from renewable feedstocks such as cheese whey permeate, cornsteep liquor, sugar beet molasses, and barley malt. Other carbon substrates may include ethanol, lactate, succinate, or glycerol.

Additionally the carbon substrate may also be one-carbon substrates such as carbon dioxide, or methanol for which metabolic conversion into key biochemical intermediates has been demonstrated. In addition to one and two carbon substrates, methylotrophic organisms are also known to utilize a number of other carbon containing compounds such as methylamine, glucosamine and a variety of amino acids for metabolic activity. For example, methylotrophic yeasts are known to utilize the carbon from methylamine to form trehalose or glycerol (Bellion et al., Microb. Growth C1 Compd., [Int. Symp.], 7th (1993), 415-32, Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Hence it is contemplated that the source of carbon utilized in the present invention may encompass a wide variety of carbon containing substrates and will only be limited by the choice of organism.

Although it is contemplated that all of the above mentioned carbon substrates and mixtures thereof are suitable in the present invention, in some embodiments, the carbon substrates are glucose, fructose, and sucrose, or mixtures of these with C5 sugars such as xylose and/or arabinose for yeasts cells modified to use C5 sugars. Sucrose may be derived from renewable sugar sources such as sugar cane, sugar beets, cassava, sweet sorghum, and mixtures thereof. Glucose and dextrose may be derived from renewable grain sources through saccharification of starch based feedstocks including grains such as corn, wheat, rye, barley, oats, and mixtures thereof. In addition, fermentable sugars may be derived from renewable cellulosic or lignocellulosic biomass through processes of pretreatment and saccharification, as described, for example, in U.S. Patent Application Publication No. 2007/0031918 A1, which is herein incorporated by reference. Biomass refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass may comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, and mixtures thereof.

In addition to an appropriate carbon source, fermentation media must contain suitable minerals, salts, cofactors, buffers and other components, known to those skilled in the art, suitable for the growth of the cultures and promotion of an enzymatic pathway described herein.

In some embodiments, the butanologen produces butanol at least 90% of effective yield, at least 91% of effective yield, at least 92% of effective yield, at least 93% of effective yield, at least 94% of effective yield, at least 95% of effective yield, at least 96% of effective yield, at least 97% of effective yield, at least 98% of effective yield, or at least 99% of effective yield. In some embodiments, the butanologen produces butanol at about 55% to at about 75% of effective yield, about 50% to about 80% of effective yield, about 45% to about 85% of effective yield, about 40% to about 90% of effective yield, about 35% to about 95% of effective yield, about 30% to about 99% of effective yield, about 25% to about 99% of effective yield, about 10% to about 99% of effective yield, or about 10% to about 100% of effective yield.

### Culture Conditions

Typically cells are grown at a temperature in the range of about 20° C. to about 40° C. in an appropriate medium. In some embodiments, the cells are grown at a temperature of 20° C., 22° C., 25° C., 27° C., 30° C., 32° C., 35° C., 37° C. or 40° C. Suitable growth media in the present invention are common commercially prepared media such as Luria Bertani (LB) broth, Sabouraud Dextrose (SD) broth or Yeast Medium (YM) broth or broth that includes yeast nitrogen base, ammonium sulfate, and dextrose (as the carbon/energy source) or YPD Medium, a blend of peptone, yeast extract, and dextrose in optimal proportions for growing most Saccharomyces cerevisiae strains. Other defined or synthetic growth media can also be used, and the appropriate medium for growth of the particular microorganism will be known by one skilled in the art of microbiology or fermentation science. The use of agents known to modulate catabolite repression directly or indirectly, e.g., cyclic adenosine 2′,3′-monophosphate (cAMP), can also be incorporated into the fermentation medium.

Suitable pH ranges for the fermentation are between pH 5.0 to pH 9.0, where pH 6.0 to pH 8.0 is preferred for the initial condition. Suitable pH ranges for the fermentation of yeast are typically between about pH 3.0 to about pH 9.0. In one embodiment, about pH 5.0 to about pH 8.0 is used for the initial condition. Suitable pH ranges for the fermentation of other microorganisms are between about pH 3.0 to about pH 7.5. In one embodiment, about pH 4.5 to about pH 6.5 is used for the initial condition.

Fermentations can be performed under aerobic or anaerobic conditions. In one embodiment, anaerobic or microaerobic conditions are used for fermentation.

In some embodiments, the culture conditions are such that the fermentation occurs without respiration. For example, cells can be cultured in a fermenter under micro-aerobic or anaerobic conditions.

### Industrial Batch and Continuous Fermentations

Butanol, or other products, can be produced using a batch method of fermentation. A classical batch fermentation is a closed system where the composition of the medium is set at the beginning of the fermentation and not subject to artificial alterations during the fermentation. A variation on the standard batch system is the fed-batch system. Fed-batch fermentation processes are also suitable in the present invention and comprise a typical batch system with the exception that the substrate is added in increments as the fermentation progresses. Fed-batch systems are useful when catabolite repression is apt to inhibit the metabolism of the cells and where it is desirable to have limited amounts of substrate in the media. Batch and fed-batch fermentations are common and well known in the art and examples can be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36:227, (1992), herein incorporated by reference.

Butanol, or other products, may also be produced using continuous fermentation methods. Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth. Continuous fermentation allows for the modulation of one factor or any number of factors that affect cell growth or end product concentration. Methods of modulating nutrients and growth factors for continuous fermentation processes as well as techniques for maximizing the rate of product formation are well known in the art of industrial microbiology and a variety of methods are detailed by Brock, supra.

It is contemplated that the production of butanol, or other products, can be practiced using batch, fed-batch or continuous processes and that any known mode of fermentation would be suitable. Additionally, it is contemplated that cells can be immobilized on a substrate as whole cell catalysts and subjected to fermentation conditions for butanol production.

Methods for Butanol Isolation from the Fermentation Medium

Bioproduced butanol may be isolated from the fermentation medium using methods known in the art for ABE fermentations (see, e.g., Durre, Appl. Microbiol. Biotechnol. 49:639-648 (1998), Groot et al., Process. Biochem. 27:61-75 (1992), and references therein). For example, solids may be removed from the fermentation medium by centrifugation, filtration, decantation, or the like. The butanol may be isolated from the fermentation medium using methods such as distillation, azeotropic distillation, liquid-liquid extraction, adsorption, gas stripping, membrane evaporation, or pervaporation.

Because butanol forms a low boiling point, azeotropic mixture with water, distillation can be used to separate the mixture up to its azeotropic composition. Distillation may be used in combination with the processes described herein to obtain separation around the azeotrope. Methods that may be used in combination with distillation to isolate and purify butanol include, but are not limited to, decantation, liquid-liquid extraction, adsorption, and membrane-based techniques. Additionally, butanol may be isolated using azeotropic distillation using an entrainer (see, e.g., Doherty and Malone, Conceptual Design of Distillation Systems, McGraw Hill, New York, 2001).

The butanol-water mixture forms a heterogeneous azeotrope so that distillation may be used in combination with decantation to isolate and purify the isobutanol. In this method, the butanol containing fermentation broth is distilled to near the azeotropic composition. Then, the azeotropic mixture is condensed, and the butanol is separated from the fermentation medium by decantation, wherein the butanol can be contacted with an agent to reduce the activity of the one or more carboxylic acids. The decanted aqueous phase may be returned to the first distillation column as reflux or to a separate stripping column. The butanol-rich decanted organic phase may be further purified by distillation in a second distillation column.

The butanol can also be isolated from the fermentation medium using liquid-liquid extraction in combination with distillation. In this method, the butanol is extracted from the fermentation broth using liquid-liquid extraction with a suitable solvent. The butanol-containing organic phase is then distilled to separate the butanol from the solvent.

Distillation in combination with adsorption can also be used to isolate butanol from the fermentation medium. In this method, the fermentation broth containing the butanol is distilled to near the azeotropic composition and then the remaining water is removed by use of an adsorbent, such as molecular sieves (Aden et al., Lignocellulosic Biomass to Ethanol Process Design and Economics Utilizing Co-Current Dilute Acid Prehydrolysis and Enzymatic Hydrolysis for Corn Stover, Report NREL/TP-510-32438, National Renewable Energy Laboratory, June 2002).

Additionally, distillation in combination with pervaporation may be used to isolate and purify the butanol from the fermentation medium. In this method, the fermentation broth containing the butanol is distilled to near the azeotropic composition, and then the remaining water is removed by pervaporation through a hydrophilic membrane (Guo et al., J. Membr. Sci. 245, 199-210 (2004)).

In situ product removal (ISPR) (also referred to as extractive fermentation) can be used to remove butanol (or other fermentative alcohol) from the fermentation vessel as it is produced, thereby allowing the microorganism to produce butanol at high yields. One method for ISPR for removing fermentative alcohol that has been described in the art is liquid-liquid extraction. In general, with regard to butanol fermentation, for example, the fermentation medium, which includes the microorganism, is contacted with an organic extractant at a time before the butanol concentration reaches a toxic level. The organic extractant and the fermentation medium form a biphasic mixture. The butanol partitions into the organic extractant phase, decreasing the concentration in the aqueous phase containing the microorganism, thereby limiting the exposure of the microorganism to the inhibitory butanol.

Liquid-liquid extraction can be performed, for example, according to the processes described in U.S. Patent Appl. Pub. No. 2009/0305370, the disclosure of which is hereby incorporated in its entirety. U.S. Patent Appl. Pub. No. 2009/0305370 describes methods for producing and recovering butanol from a fermentation broth using liquid-liquid extraction, the methods comprising the step of contacting the fermentation broth with a water immiscible extractant to form a two-phase mixture comprising an aqueous phase and an organic phase. Typically, the extractant can be an organic extractant selected from the group consisting of saturated, mono-unsaturated, poly-unsaturated (and mixtures thereof) C₁₂ to C₂₂ fatty alcohols, C₁₂ to C₂₂ fatty acids, esters of C₁₂ to C₂₂ fatty acids, C₁₂ to C₂₂ fatty aldehydes, and mixtures thereof. The extractant(s) for ISPR can be non-alcohol extractants. The ISPR extractant can be an exogenous organic extractant such as oleyl alcohol, behenyl alcohol, cetyl alcohol, lauryl alcohol, myristyl alcohol, stearyl alcohol, 1-undecanol, oleic acid, lauric acid, myristic acid, stearic acid, methyl myristate, methyl oleate, undecanal, lauric aldehyde, 20-methylundecanal, and mixtures thereof.

In some embodiments, an alcohol ester can be formed by contacting the alcohol in a fermentation medium with an organic acid (e.g., fatty acids) and a catalyst capable of esterfiying the alcohol with the organic acid. In such embodiments, the organic acid can serve as an ISPR extractant into which the alcohol esters partition. The organic acid can be supplied to the fermentation vessel and/or derived from the biomass supplying fermentable carbon fed to the fermentation vessel. Lipids present in the feedstock can be catalytically hydrolyzed to organic acid, and the same catalyst (e.g., enzymes) can esterify the organic acid with the alcohol. Carboxylic acids that are produced during the fermentation can additionally be esterified with the alcohol produced by the same or a different catalyst. The catalyst can be supplied to the feedstock prior to fermentation, or can be supplied to the fermentation vessel before or contemporaneously with the supplying of the feedstock. When the catalyst is supplied to the fermentation vessel, alcohol esters can be obtained by hydrolysis of the lipids into organic acid and substantially simultaneous esterification of the organic acid with butanol present in the fermentation vessel. Organic acid and/or native oil not derived from the feedstock can also be fed to the fermentation vessel, with the native oil being hydrolyzed into organic acid. Any organic acid not esterified with the alcohol can serve as part of the ISPR extractant. The extractant containing alcohol esters can be separated from the fermentation medium, and the alcohol can be recovered from the extractant. The extractant can be recycled to the fermentation vessel. Thus, in the case of butanol production, for example, the conversion of the butanol to an ester reduces the free butanol concentration in the fermentation medium, shielding the microorganism from the toxic effect of increasing butanol concentration. In addition, unfractionated grain can be used as feedstock without separation of lipids therein, since the lipids can be catalytically hydrolyzed to organic acid, thereby decreasing the rate of build-up of lipids in the ISPR extractant.

In situ product removal can be carried out in a batch mode or a continuous mode. In a continuous mode of in situ product removal, product is continually removed from the reactor. In a batchwise mode of in situ product removal, a volume of organic extractant is added to the fermentation vessel and the extractant is not removed during the process. For in situ product removal, the organic extractant can contact the fermentation medium at the start of the fermentation forming a biphasic fermentation medium. Alternatively, the organic extractant can contact the fermentation medium after the microorganism has achieved a desired amount of growth, which can be determined by measuring the optical density of the culture. Further, the organic extractant can contact the fermentation medium at a time at which the product alcohol level in the fermentation medium reaches a preselected level. In the case of butanol production according to some embodiments of the present invention, the organic acid extractant can contact the fermentation medium at a time before the butanol concentration reaches a toxic level, so as to esterify the butanol with the organic acid to produce butanol esters and consequently reduce the concentration of butanol in the fermentation vessel. The ester-containing organic phase can then be removed from the fermentation vessel (and separated from the fermentation broth which constitutes the aqueous phase) after a desired effective titer of the butanol esters is achieved. In some embodiments, the ester-containing organic phase is separated from the aqueous phase after fermentation of the available fermentable sugar in the fermentation vessel is substantially complete.

### Confirmation of Isobutanol Production

The presence and/or concentration of isobutanol in the culture medium can be determined by a number of methods known in the art (see, for example, U.S. Pat. No. 7,851,188, incorporated by reference). For example, a specific high performance liquid chromatography (HPLC) method utilizes a Shodex SH-1011 column with a Shodex SHG guard column, both may be purchased from Waters Corporation (Milford, Mass.), with refractive index (RI) detection. Chromatographic separation is achieved using 0.01 M H₂SO₄ as the mobile phase with a flow rate of 0.5 mL/min and a column temperature of 50° C. Isobutanol has a retention time of 46.6 min under the conditions used.

Alternatively, gas chromatography (GC) methods are available. For example, a specific GC method utilizes an HP-INNOWax column (30 m×0.53 mm id, 1 μm film thickness, Agilent Technologies, Wilmington, Del.), with a flame ionization detector (FID). The carrier gas is helium at a flow rate of 4.5 mL/min, measured at 150° C. with constant head pressure; injector split is 1:25 at 200° C.; oven temperature is 45° C. for 1 min, 45 to 220° C. at 10° C./min, and 220° C. for 5 min; and FID detection is employed at 240° C. with 26 mL/min helium makeup gas. The retention time of isobutanol is 4.5 min.

While various embodiments of the present invention have been described herein, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the claims and their equivalents.

All publications, patents, and patent applications mentioned in this specification are indicative of the level of those skilled in the art to which this invention pertains, and are herein incorporated by reference to the same extent as if each individual publication, patent, or patent application was specifically and individually indicated to be incorporated by reference.

### EXAMPLES

The present invention is further defined in the following Examples. It should be understood that these Examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### General Methods

Standard recombinant DNA, molecular cloning techniques and transformation protocols used in the Examples are well known in the art and are described by Sambrook et al. (Sambrook, J., Fritsch, E. F. and Maniatis, T. (Molecular Cloning: A Laboratory Manual; Cold Spring Harbor Laboratory Press, Cold Spring Harbor, 1989, here in referred to as Maniatis), by Ausubel et al. (Ausubel et al., Current Protocols in Molecular Biology, pub. by Greene Publishing Assoc. and Wiley-Interscience, 1987) and by Amberg et al (Amberg, D.C., Burke, D. J. and Strathern, J. N. (Methods in Yeast Genetics: A Cold Spring Harbor Laboratory Course Manual, Cold Spring Harbor Press, 2005). Materials and methods suitable for the maintenance and growth of bacterial cultures are well known in the art. Techniques suitable for use in the following examples may be found as set out in Manual of Methods for General Bacteriology (Phillipp et al., eds., American Society for Microbiology, Washington, D.C., 1994) or by Thomas D. Brock in (Brock, Biotechnology: A Textbook of Industrial Microbiology, Second Edition, Sinauer Associates, Inc., Sunderland, Mass. (1989). All reagents, restriction enzymes and materials used for the growth and maintenance of bacterial cells were obtained from Sigma-Aldrich Chemicals (St. Louis, Mo.), BD Diagnostic Systems (Sparks, Md.), Invitrogen (Carlsbad, Calif.), HiMedia (Mumbai, India), SD Fine chemicals (India), or Takara Bio Inc. (Shiga, Japan), unless otherwise specified.

The meaning of abbreviations is as follows: “sec” means second(s), “min” means minute(s), “h” means hour(s), “nm” means nanometers, “uL” means microliter(s), “mL” means milliliter(s), “mg/mL” means milligram per milliliter, “L” means liter(s), “nm” means nanometers, “mM” means millimolar, “M” means molar, “mmol” means millimole(s), “μmole” means micromole(s), “kg” means kilogram, “g” means gram(s), “μg” means microgram(s) and “ng” means nanogram(s), “PCR” means polymerase chain reaction, “OD” means optical density, “OD600” means the optical density measured at a wavelength of 600 nm, “kDa” means kilodaltons, “g” can also mean the gravitation constant, “bp” means base pair(s), “kbp” means kilobase pair(s), “kb” means kilobase, “%” means percent, “% w/v” means weight/volume percent, “% v/v” means volume/volume percent, “HPLC” means high performance liquid chromatography, “g/L” means gram per liter, “μg/L” means microgram per liter, “ng/μL” means nanogram per microliter, “pmol/μL” means picomol per microliter, “RPM” means rotation per minute, “μmol/min/mg” means micromole per minute per milligram, “w/v” means weight per volume, “v/v” means volume per volume.

### Example 1a

### Distillation of Isobutanol/Isobutyric Compositions Results in No Reduction in Isobutyric Acid Activity

Into a glass vessel were mixed 150 g of deionized water, 5 g of isobutanol (Sigma-Aldrich reagent grade, St. Louis, Mo.) and 1 g of isobutyric acid (Sigma-Aldrich reagent grade). A clear colorless homogeneous solution was obtained with a measured pH of 3. Heat was applied externally via a mantle to bring the solution to a steady boil starting at 94.8° C. The vapors were condensed and the liquid condensate was collected in a graduated cylinder. The temperature of the boiling solution rose to 99.7° C., and a total of 9.5 ml of liquid distillate were collected over the course of the experiment. The liquid distillate partitioned into 2.5 ml of an aqueous layer and 7 ml of an organic layer. The pH of the aqueous portion of the distillate was found to be 4.5. The organic portion of the distillate was analyzed and found to contain 78.8 wt % isobutanol, 1.1 wt % isobutyric acid and 20.1 wt % water.

### Example 1b

### Reduction of Isobutyric Acid Activity by Titration of Example 1a Distillate

Dilute ammonium hydroxide solution containing 5 wt % NH₃ was added dropwise to the liquid distillate collected in Example 1a until the pH of the aqueous portion was raised to 9. The aqueous and organic phases were allowed to reach equilibrium. The organic portion of the titrated distillate was analyzed and found to contain 78.9 wt % isobutanol, 0.1 wt % isobutyric acid and 21.0 wt % water.

### Example 1c

### Reduction of Isobutyric Acid Activity Before Distillation

Into a glass vessel were mixed 150 g of deionized water, 5 g of isobutanol (Sigma-Aldrich reagent grade) and 1 g of isobutyric acid (Sigma-Aldrich reagent grade). A clear colorless homogeneous solution was obtained with a measured pH of 3. The solution was titrated with 5 g of a dilute ammonium hydroxide solution containing 5 wt % NH₃ and this brought the pH up to 8. Heat was applied externally via a mantle to bring the solution to a steady boil starting at 95.1° C. The vapors were condensed and the liquid condensate was collected in a graduated cylinder. The temperature of the boiling solution rose to 100.2° C., and a total of 8.5 ml of liquid distillate were collected over the course of the experiment. The liquid distillate partitioned into 2 ml of an aqueous layer and 6.5 ml of an organic layer. The pH of the aqueous portion of the distillate was found to be 10. The organic portion of the distillate was analyzed and found to contain 79.2 wt % isobutanol, 20.8 wt % water and no detectable isobutyric acid.

### Example 2a

### Reduction in Isobutyric Acid Activity by Addition of Neutralizing Agent

To determine the effect of sodium and potassium carbonate on an aqueous liquid phase composition comprising isobutyric acid, solutions of sodium carbonate (0, 1, 3, and 10%), potassium carbonate (0, 1, 3, and 10%), and isobutyric acid (0, 1, 3, and 10 g/L) were made. To determine the effect of sodium carbonate (Sigma, St. Louis, Mo.) and potassium carbonate (Fisher, Pittsburgh, Pa.) solutions on isobutyric acid (Sigma) concentrations, alkali titrations were performed. Briefly, increasing concentrations of each carbonate solution was mixed with a fixed level of isobutyric acid (0, 1, 3, or 10 g/L) at room temperature for at least one hour. After the solutions were mixed, samples from the mixture were analyzed by gas chromatography (GC) as well as for total acidity. Results of the total acid titrations are shown in Table 1. As demonstrated in the table, both sodium and potassium carbonate solutions were capable of reducing the acidity of an aqueous liquid phase composition comprising isobutyric acid.

TABLE 1
Sodium and potassium carbonate effect on isobutyric acid concentration.

Feed Isobutyric Acdi
Concentration (g/L)

| | 0 | 1 | 3 | 10 |
|---|---|---|---|---|
| Sodium Carbonate | | | | |
| Conc. (%) | | | | |
| 0 | 0 | 1 | 2.8 | 10.1 |
| 1 | 0 | 0.03 | 1.2 | 8.5 |
| 3 | 0 | 0 | 0.2 | 5.8 |
| 10 | 0 | 0 | 0 | 0.5 |
| Potassium Carbonate | | | | |
| Conc. (%) | | | | |
| 0 | 0 | 1 | 3.1 | 9.6 |
| 1 | 0 | 0 | 1.8 | 9.1 |
| 3 | 0 | 0 | 0.4 | 6.7 |
| 10 | 0 | 0 | 0 | 1.4 |

### Example 2b

### Reduction in Isobutyric Acid Activity by Addition of Neutralizing Agent Prophetic

At the end of the fermentation process, a fermentation broth is sampled in a 10 ml falcon tube. From the fermentation broth, 3 samples per condition are taken and placed into tubes. The samples are spun down at room temperature for 2 minutes. Three tubes representing one experimental condition are treated with a neutralizing agent (e.g., sodium carbonate). The samples are vortexed and allowed to sit for 60 minutes at room temperature. Three tubes representing the control are not treated or vortexed and are allowed to sit for 60 minutes at room temperature. The supernatant from these samples is decanted into spin filters. The resulting filtrate is analyzed for both the “treated” sample as well as the “untreated” sample. The supernatant samples are analyzed via a GC method as well as total acidity to determine the untreated and treated concentrations of isobutyric acid and total acid.

### Example 3

### Increased Liquid-Liquid Extraction Efficiency of Carboxylic Acids from Aqueous Broth into Extractant at Lower pH

Efficiency of liquid-liquid extraction of several specific carboxylic acids from aequeous broth into a lipophilic secondary phase consisting of an extractant (e.g., oleyl alcohol, corn oil fatty acids (COFA), soy oil fatty acids (SOFA), and oleic acid) was investigated at different pH values. To investigate the effect of pH on extraction efficiency of carboxylic acids with different extractants, 2 ml of fermentation medium were placed in 15 ml centrifugation tubes (fermentation medium: 6.7 g/l, Yeast Nitrogen Base w/o amino acids (Difco 0919-15-3), 10 mg/L thiamine, 10 mg/L niacine, 3.5 mL/L of ethanol, 36 mL/L of 50% w/w glucose, 1 mL/L of Tween & ergosterol solution, 100 ml/L 1M MES buffer at pH=5.5, 1.4 g/L of Y2001 Sigma Yeast Synthetic Drop-out Medium Supplements, 76 mg/L histidine, 76 mg/L leucine, 76 mg/L tryptophan and 76 mg/L uracil). Carboxylic acids were added to the sample broth at approximate initial concentrations of 168 mM for acetic acid; 20 mM for formic acid; 72 mM for hydroxymethylbutyric acid (HMB); and 94 mM for isobutyric acid. One set of centrifugation tubes was adjusted to low (pH=3.14), another to a higher (pH=4.94), and yet another to an even higher pH (pH=7.24), as determined in the aqueous broth at room temperature (RT). Subsequently, the aqueous sample mixtures were either kept without addition of ISPR extractant, or overlaid with either 2 or 6 ml of oleyl alcohol and rigorously vortexed for 5 minutes. The samples were subsequently incubated in a shaker at 30° C. and 300 rpm for 30 minutes. From each of the sample tubes, one milliliter of aqueous (/oleyl alcohol) sample mixture was withdrawn into an Eppendorf tube and centrifuged at RT at 16.000 rpm for 5 minutes. Finally, the aqueous broth samples were withdrawn from the upper section of the bottom phase for further analysis. These aqueous broth samples were filtered with a 0.20 μm filter (Nanosep MF, Pall) and analysed by HPLC. A Biorad Aminex HPX-87H column was used in an isocratic method with 0.01N sulfuric acid as eluent on a Waters Alliance 2695 Separations Module (Milford, Mass.). The flow rate was 0.60 ml/min, the column temperature 40° C., the injection volume 10 ul and the run time was 58 minutes. Detection was carried out with a refractive index detector (Waters 2414 RI) operated at 40° C. and an UV detector (Waters 2996 PDA) at 210 nm. Lower pH allows for higher extraction of the respective carboxylic acid from the broth into the hydrophobic ISPR extractant (Tables 2-5).

TABLE 2
Increased extraction efficiency of carboxylic acid with
oleyl alcohol in fermentation medium with lower pH.

| | ISPR ratio (Aq):(Oleyl Alcohol) | | | ISPR ratio (Aq):(Oleyl Alcohol) | | | ISPR ratio (Aq):(Oleyl Alcohol) | | |
|---|---|---|---|---|---|---|---|---|---|
| | 1:0 | 1:1 | 1:3 | 1:0 | 1:1 | 1:3 | 1:0 | 1:1 | 1:3 |
| pH | Conc. Isobutyric Acid (mM) | | | Conc. HMB (mM) | | | Conc. Acetic Acid (mM) | | |
| 7.24 | 93.79 | 93.36 | 91.95 | 71.33 | 72.46 | 73.31 | 165.56 | 169.15 | 173.63 |
| 4.94 | 94.34 | 46.14 | 30.83 | 72.41 | 72.00 | 71.86 | 167.47 | 162.73 | 159.23 |
| 3.14 | 94.37 | 19.77 | 8.10 | 72.13 | 54.07 | 40.28 | 167.70 | 140.04 | 106.43 |

TABLE 3
Increase extraction efficiency of carboxylic acid with SOFA in
fermentation medium with lower pH.

| | ISPR ratio (Aq):(SOFA) | | |
|---|---|---|---|
| | 1:0 | 1:1 | 1:3 |
| pH | Conc. Isobutyric Acid (%) | | |
| 3.93 | 100.00 | 31.37 | 12.68 |
| 4.92 | 100.00 | 53.76 | 27.63 |
| 6.64 | 100.00 | 74.48 | 33.23 |

TABLE 4
Increased extraction efficiency of carboxylic acid with oleic
acid in fermentation medium with lower pH.

| | ISPR ratio (Aq):(Oleic Acid) | | |
|---|---|---|---|
| | 1:0 | 1:1 | 1:3 |
| pH | Conc. Isobutyric Acid (%) | | |
| 4.08 | 100.00 | 29.60 | 13.00 |
| 4.82 | 100.00 | 49.16 | 23.36 |
| 6.95 | 100.00 | 67.35 | 28.60 |

TABLE 5
Increased extraction efficiency of carboxylic acid with COFA
in fermentation medium with lower pH.

| | ISPR ratio (Aq):(COFA) | | |
|---|---|---|---|
| | 1:0 | 1:1 | 1:3 |
| pH | Conc. Isobutyric Acid (%) | | |
| 4.08 | 100.00 | 29.81 | 12.81 |
| 4.82 | 100.00 | 52.89 | 30.00 |
| 6.95 | 100.00 | 70.40 | 31.27 |

### Example 4

### Reactive Liquid-Liquid Extraction of Carboxylic Acids from Aqueous Solution into Oleyl Alcohol (OA) and Reactive Extractant Mixtures

An aqueous solution with about 12.5 mM of isobutanol, isobutyric acid (free acid), formic acid (from sodium formate) and acetic acid (from sodium acetate) was prepared in a 100 mM MES and 25 mM phosphate buffer at pH=5.3 and T=30° C. Subsequently 2 mL of the compound solution was transferred to 15 mL centrifuge tubes and overlaid with 2 mL, 6 mL or 10 mL of either OA, or OA containing approximately 100 mM of tri-n-butyl phosphate (TBP), tru-n-octylamine (TOA), or Aliquat 336 (A336), respectively. The emulsions were rigorously vortexed and incubated at 30° C. and 300 rpm for 30 minutes. The emulsions were rigorously vortexed again, 1 mL sample was withdrawn and centrifuged in an Eppendorf table top centrifuge for 5 minutes at 16,000 rpm. Samples were taken from the top (extractant) as well as bottom (aqueous broth) phase and stored in the freezer at −20° C. for further analysis.

Aqueous broth samples were filtered through a 0.20 μm filter (Nanosept MF, Pall) and analyzed by HPLC. For HPLC analysis of aqueous broth a Biorad Aminex HPX-87H column was used in an isocratic method with 0.01 N sulfuric acid as eluent on a Waters Alliance 2695 Separations Module (Milford, Mass.). The flow rate was 0.08 ml/min, column temperature was 60° C., the injection volume was 10 ul and the sample run time was 38 minutes. Detection was carried out with a refractive index detector (Waters 2414 RI) operated at 50° C. and an UV detector (Waters 2996 PDA) at 210 nm.

Initial aqueous concentrations and aqueous concentrations after addition of 2, 6 and 10 mL of either OA, OA & tributyl phosphate, OA & trioctyl amine, and OA & Aliquat 336 are given in Table 6.

Water extracted into the oleyl alcohol was found to be 10 mL per liter of oleyl alcohol, corresponding to 1.0% (v/v). In subsequent calculations, the volume of water was corrected for the extracted volume, but no volume increase of the diluent was considered.

The partition coefficient of isobutanol into oleyl alcohol was determined to be 2.97 mol/mol. For isobutyric acid, acetic acid, formic acid, H₂PO₄⁻ and MES (2-(N-morpholino)ethanesulfonic acid), pKₐ values of 4.84, 4.75, 3.75, 7.21 and 6.15 were assumed (Table 7). Partitioning of the deprotonated form of the acid was neglected, and only the protonated form was assumed to transition into the diluent oleyl alcohol. For isobutyric acid, acetic acid, formic acid, H₂PO₄⁻ and MES (2-(N-morpholino)ethanesulfonic acid), partition coefficients on a mol/mol base of 2.97, 1.91, 0.08, 0.00, 0.00 and 0.00 were found, respectively (Table 7).

Partitioning and/or reactive extraction of isobutanol and acids of the compound solution into oleyl alcohol containing 100 mM TPO, TOA or A336 was analyzed.

The mass law equilibria describing the (reactive) extraction of a respective compound (C) in aqueous phase by an extractant in the organic phase (E) as a compound-extractant complex into the diluent (C:E) assuming a stoichiometry of 1:1 can be described according to:

$${[ C ]}_{aq} + {[ E ]}_{org} {}^{{K}_{E}} {[ C \text{:} E ]}_{org}$$

The equilibrium extraction constant K_{E} can be derived accordingly as

$${K}_{E} = \frac{{[ C \text{:} E ]}_{org}}{{[ C ]}_{aq} \cdot {[ E ]}_{org}}$$

While [C]_{aq} was measured, the concentration of free extractant [E]_{org} in the diluent had to be calculated according to

$${[ E ]}_{org} = [ {E}_{0} ] - {\sum }_{i = 1}^{n} {[ {C}_{i} \text{:} E ]}_{org}$$

with E₀ designating the initial concentration of the extractant in the diluent (in our case 100 mM), less the concentrations of all compound-extractant complexes formed for all n compounds in the compound solution. Furthermore, it can be assumed that complex formation of the extractive amines used in this study mainly occur with the deprotonated acid species, consequently interactions of the extractant with the protonated species HA of an acid were not considered, and [C]_{aq} was used equivalent to [A⁻]_{aq}.

As a second parameter, the apparent partitioning coefficient Pₐₚₚ was calculated. Pₐₚₚ characterizes the concentration of the compound in the organic phase as compared to the aqueous phase, according to

$${P}_{app} = \frac{{[ C ]}_{org} + {[ C \text{:} E ]}_{org}}{{[ C ]}_{aq}}$$

Results of the analysis are given in Table 8. It can be seen that the partitioning coefficient of isobutanol into the diluent was not influenced by the addition of the extractant, with a partitioning coefficient of 3.08, 2.93 and 2.93 in oleyl alcohol with 100 mM of tibutyl phosphate, trioctyl amine and Aliquat 336, respectively (Table 8). The partitioning coefficient of isobutanol in oleyl alcohol was determined to be 2.97 (Table 7).

Extraction of isobutyric acid was most efficient with A336, less efficient with TOA, and least efficient with TBP. Other carboxylic acids were extracted with the same efficiency as isobutyric acid in A336, TOA, and TBP. Acetic acid and formic acid were extracted less efficiently than isobutyric acid; however, and H₂PO₄⁻ is hardly extracted at all. Overall, Aliquat 336 exhibits not only high reactive extraction properties for isobutyric acid, but also a reasonable selectivity towards isobutyric acid as compared to the other organic acids and especially towards H₂PO₄⁻.

TABLE 6
Initial aqueous concentrations of compounds in the compound solution (“aqueous solution”) and
aqueous concentrations after addition of different volumes (2 mL, 6 mL or 10 mL) of OA or OA
containing about 100 mM of tributyl phosphate, trioctyl amine and Aliquat 336, respectively.

| | Volume | Isobutanol | Isobutyric | Acetic | Formic | Phosphate | MES |
|---|---|---|---|---|---|---|---|
| Composition | (mL) | (mM) | Acid (mM) | Acid (mM) | Acid (mM) | (AU) | (AU) |
| Aqueous | 2 | 11.95 | 12.09 | 13.29 | 11.65 | 648363 | 6874000 |
| OA | 2 | 3.01 | 8.01 | 13.13 | 11.96 | 656297 | 6954908 |
| | 6 | 1.19 | 4.95 | 12.98 | 12.68 | 673645 | 7135183 |
| | 10 | 0.77 | 3.57 | 12.73 | 13.05 | 676874 | 7168671 |
| OA + TBP | 2 | 2.86 | 7.78 | 13.14 | 11.98 | 657205 | 6961921 |
| | 6 | 1.14 | 4.86 | 12.93 | 12.67 | 672453 | 7126032 |
| | 10 | 0.76 | 3.60 | 12.70 | 13.13 | 678777 | 7192123 |
| OA + TOA | 2 | 3.11 | 5.00 | 12.03 | 10.86 | 653451 | 6946798 |
| | 6 | 1.20 | 2.66 | 10.32 | 10.10 | 660520 | 7048354 |
| | 10 | 0.77 | 1.83 | 9.55 | 9.82 | 670916 | 7147700 |
| OA + A336 | 2 | 2.99 | 2.83 | 8.84 | 6.30 | 633289 | 6670682 |
| | 6 | 1.23 | 1.24 | 6.16 | 3.99 | 620318 | 6671910 |
| | 10 | 0.78 | 0.79 | 4.89 | 3.15 | 612705 | 6836766 |

TABLE 7
Use pKa and P[A−] values, and experimentally determined
partition coefficients for isobutanol and the protonated acids.

| | | Partition coefficients (P) | |
|---|---|---|---|
| | pKa | [HA] org/aq [mol/mol] | [A−] org/aq [mol/mol]¹ |
| Isobutanol | — | 2.97 | |
| Isobutyric acid | 4.84 | 1.91 | 0.00 |
| Acetic Acid | 4.75 | 0.08 | 0.00 |
| Formic Acid | 3.75 | 0.00 | 0.00 |
| H₂PO₄⁻ | 7.21 | 0.00 | 0.00 |
| MES | 6.15 | 0.00 | 0.00 |

¹Assumed fixed values

TABLE 8
Equilibrium extraction constant KE and (apparent) partitioning coefficient
of investigated compounds in oleyl alcohol with 100 mM of tributyl
phosphate, trioctyl amine and Aliquat 336, respectively.

| | +TBA | | +TOA | | +A336 | |
|---|---|---|---|---|---|---|
| | K_{E} | Pₐₚₚ⁽¹⁾ | K_{E} | Pₐₚₚ⁽¹⁾ | K_{E} | Pₐₚₚ⁽¹⁾ |
| Isobutanol | — | 3.08 | — | 2.93 | — | 2.93 |
| Isobutyric | 0.000 | 0.57 | 0.010 | 1.44 | 0.036 | 3.31 |
| Acid | | | | | | |
| Acetic Acid | 0.000 | 0.00 | 0.001 | 0.02 | 0.005 | 0.52 |
| Formic Acid | 0.000 | 0.02 | 0.001 | 0.12 | 0.007 | 0.87 |
| H₂PO₄⁻ | 0.000 | 0.00 | 0.003 | 0.00 | 0.000 | 0.02 |

⁽¹⁾apparent partitioning coefficient Pₐₚₚ is determined under addition of diluent with 100 mM extractant to the aqueous compound solution at a ratio of 1:1.

### Example 5

### Reactive Liquid-Liquid Extraction of Carboxylic Acids Produced from a Biobutanol Process into Oleyl Alcohol and Reactive Extractant Mixtures

An isobutanologen was cultivated in 20 ml of corn mash in 125 ml shake flasks with a simultaneous saccharification and fermentation (SSF) process. Briefly, a 125 ml aerobic shake flask was prepared with 10 ml seed medium (50% yeast synthetic medium w/o amino acids and w/o glucose (2×); 10% 2× supplement a.a. solution without histidine and uracil (SAAS-1 10×); 0.35% ethanol stock solution; 1.8% 50% w/w glucose stock solution; and 37.85% bidest H₂O to a total of 10 mL) and inoculated with a vial of frozen glycerol stock culture of PNY 2242. The culture was incubated at 30° C. and 250 rpm for 24 hours in an Innova Laboratory Shaker (New Brunswick Scientific, Edison, N.J.). Subsequently 3 ml of the isobutanologen seed culture were transferred each into 2×2000 mL aerobic shake flasks filled with 100 ml STAGE 1 medium (50% yeast synthetic medium w/o amino acids and w/o glucose (2×); 10% 2× supplement a.a. solution without histidine and uracil (SAAS-1 10×); 0.35% ethanol stock solution; 1.8% 50% w/w glucose stock solution; and 37.85% bidest H₂O to a total of 10 mL). Resulting stage 2 cultures with total culture volume of 103 ml were incubated again at 250 rpm for 24 hours. Next the two 2 L flasks were combined and proper culture volume distributed into seven 50 ml centrifuge tubes to each yield an OD of 2 in 20 ml production media (169.4 ml CCM (centrifuged corn mash); 0.8 ml urea stock solution; 0.6 ml nicotinic acid (10 g/L); 0.6 ml thiamine (10 g/L); 1.2 ml ethanol; 7.2 ml 50% glucose solution; 0.2 ml Ergosterol & Tween; 20 ml 1M MES buffer). Cultures were spun down at 9500 rpm for 20 minutes and supernatants were discarded. Cell pellet was re-suspended into 20 ml of production medium in the tubes. 20 ml of solvent was added to seven 125 ml shake flask according to Table 9:

TABLE 9
Experimental design

| Flask | Oleyl alcohol | Aliquat | Trioctylamine |
|---|---|---|---|
| [no] | [% vol/vol] | [% vol/vol] | [% vol/vol] |
| 1 | 100 | | |
| 2 | 50 | 50 | |
| 3 | 80 | 20 | |
| 4 | 95 | 5 | |
| 5 | 50 | | 50 |
| 6 | 80 | | 20 |
| 7 | 95 | | 5 |

The 20 ml cell cultures in the tubes were transferred into the shake flasks pre-filled with solvent. 50 μl of distillase added, and the shake flasks were closed with a close lid (for low oxygen/anaerobic cultivation). The flasks were subsequently incubated at 30° C. and 250 rpm for 48 hours. Each flask was sampled twice a day to measure glucose via YSI. After 48 hours, 2 ml aqueous and 1 ml of solvent phase were saved for exo-metabolite, starch and extractant analysis.

Extracellular compound analysis in supernatant was accomplished by HPLC. A Biorad Aminex HPX-87H column was used in an isocratic method with 0.01N sulfuric acid as eluent on a Waters Alliance 2695 Separations Module (Milford, Mass.). Flow rate was 0.60 ml/min, column temperature 40° C., injection volume 10 ul and run time 58 min. Detection was carried out with a refractive index detector (Waters 2414 RI) operated at 40° C. and an UV detector (Waters 2996 PDA) at 210 nm.

For starch analysis, culture samples were centrifuged for 5 minutes at 14,000 rpm and solvent atop the aqueous layer was removed. Remaining aqueous fraction was re-suspended, thoroughly mixed and 0.30 g of the mixture transferred into previously weighted vials. Subsequently 900 μl of α-amylase solution in pH=7 MOPS buffer (10.26 g/L MOPS (Sigma-Aldrich No. M-1254); 0.74 g/L calcium chloride dihydrate) (177 mg/L α-amylase from Sigma-Aldrich, No. A4551) was added and samples were incubated in a thermomixer at 90° C. and 1100 rpm for 75 minutes. After cooling to room temperature 600 μl of amyloglucosidase solution (1068 mg/L amyloglucosidase (Roche, No. 11202367001) in sodium acetate buffer, pH=4.5) was added and the samples again incubated in a thermomixer at 55° C. and 1100 rpm for 15 hours. Finally the samples were incubated for 5 minutes at 99° C. and 1100 rpm, transferred to previously weighted 10 mL volumetric flasks, filled at level with water, weights were recorded, 400 μl of the solution filtered through a centrifugal filter and subjected to HPLC analysis.

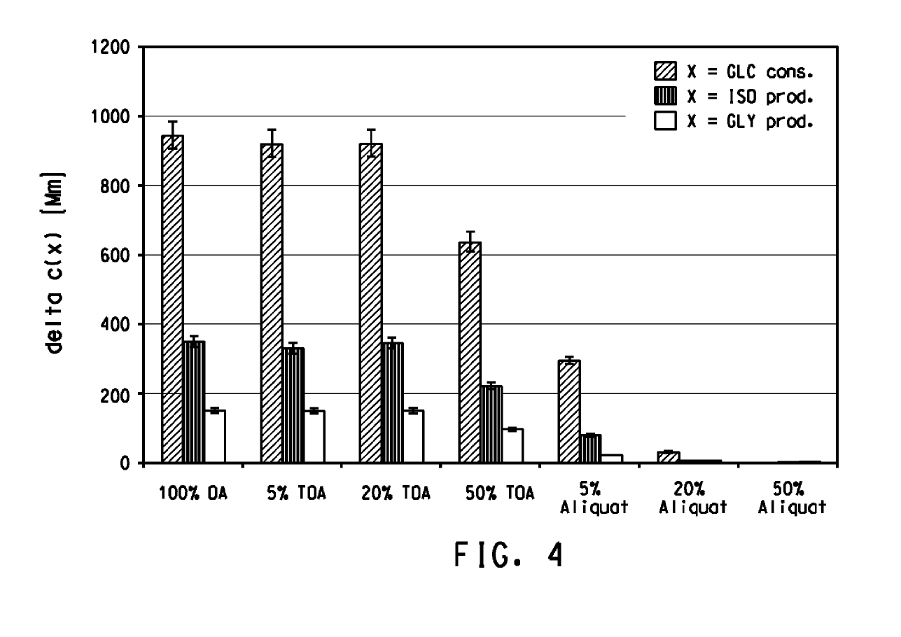

The cultures contained a second layer with either oleyl alcohol (OA) or an OA phase containing different portions of either trioctylamine (TOA) or aliquate for liquid-liquid extraction (LLX). Final effective concentrations of isobutanol and glycerol produced as well as glucose consumed as determined from analysis of both phases are depicted in FIG. 4. It can be seen that while the culture with only OA as LLX extractant performed best, OA-TOA mixtures of 95:5 and 80:20 (v/v) exhibited lower glucose consumption as well as production of isobutanol. Further, an OA:TOA ratio of 50:50 reduced performance of the butanologen. All three OA:Aliquat ratios, 95:5, 80:20 and 50:50 yielded lower performance (FIG. 4).

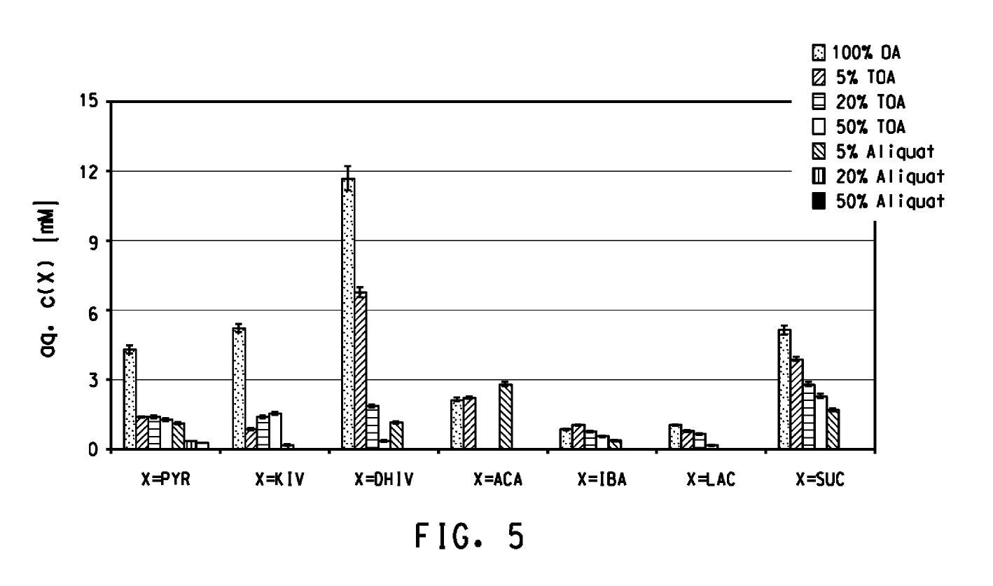

Aqueous concentrations of selected carboxylic acids were quantified at the end of the 48 hour fermentation run. Concentrations of most of the organic acid concentrations were significantly higher in the culture with only OA as extractant than on mixtures of OA with either TOA or Aliquat (FIG. 5).

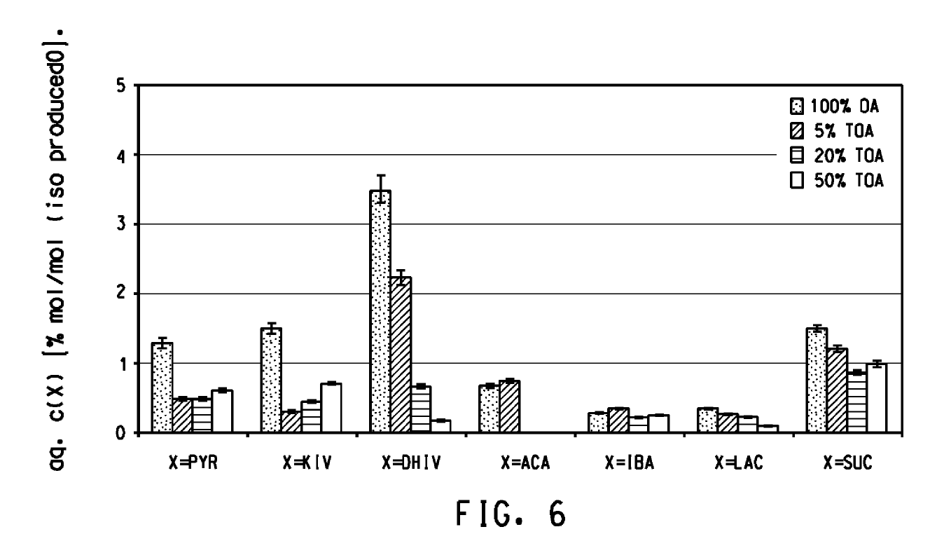

If (i) yield of isobutanol and other organic acids on glucose is assumed to be constant, and (ii) the partition coefficient of the respective organic acids into the extractant mixture is assumed to be similar, the aqueous concentration of the organic acids given as a percentage of the produced isobutanol should be the same. However, analysis of the OA and OA:TOA performing cultures indicated that the % concentration of most of the organic acids in the OA:TOA mixtures were lower than in the OA only culture, and the aqueous concentration decreased with increasing fraction of TOA in the extractant (FIG. 6).

pH at the end of the fermentation run was analyzed for the cultures. Initial pH of the buffer in the cultures was expected to be 5.50. The lowest final pH was found in the OA cultures and pH increased with increasing fractions of TOA in the ISPR solvent (Table 10). The free glucose concentration measured during the SSF was indicative of the glucose consumption rate. The OA cultures demonstrated the lowest glucose concentrations at the same distillase loading, which indicated the highest volumetric glucose consumption of the cultures (Table 10).

TABLE 10
Measured concentrations of free glucose during the SSF as determined
by the bioanalyzer, as well as pH determined at the end of the
experiment after elapsed process time (EPT) of 48 hours.

| | C (g/c) | | C (g/c) | | |
|---|---|---|---|---|---|
| | [g/L] | C (g/c) [g/L] | [g/L] | C (g/c) [g/L] | pH |
| EPT (h) | 3.67 | 21.00 | 28.00 | 48.00 | 48.00 |
| OA | 37.2 | 34.60 | 29.7 | 1.20 | 4.36 |
| OA + 5% TOA | 42.1 | 44.20 | 38.4 | 2.04 | 4.98 |
| OA + 20% TOA | 39.1 | 41.50 | 34.5 | 2.49 | 5.30 |
| OA + 50% TOA | 39.6 | 45.50 | 36.4 | 25.90 | 5.42 |
| OA + 5% aliquate | 38.9 | 52.90 | 53.1 | 62.40 | 4.64 |
| OA + 20% | 41.9 | 59.80 | 67.5 | 82.60 | 4.83 |
| aliquate | | | | | |
| OA + 50% | 40.9 | 62.00 | 65.1 | 83.70 | 4.63 |
| aliquate | | | | | |

### Example 6

### Degradation of Isobutyric Acid as Evidenced by Anaerobic Gas Production

To determine if isobutyric acid is anaerobically degradable and whether it could inhibit degradation of a known biodegradable substrate, Biological Methane Potential (BMP) and Acetate Toxicity Assays (ATA) tests were performed using an anaerobic inoculum taken from an anaerobic reactor that treats evaporate condensates. For the ATA tests, the seed inoculum was fed a known biodegradable synthetic feed composed of acetate and propionate with increasing levels of isobutyric acid (iBA). For the BMP tests, iBA provided the sole source of substrate over a range of conditions. Feed controls were prepared at organic loadings that spanned the lower and upper range of loadings used in the ATA and BMP tests, and gas production was monitored.

Experimental samples were prepared as shown in Table 11. Samples were prepared in triplicate with the seed concentration for all test bottles being 2000 mg/L. The feed control and ATA biodegradable synthetic feed composition contained 4000 mg/L acetate and 2000 mg/L propionate. The inorganic nutrient media used was similar to to media presented in Table 5.1 of Speece, Anaerobic Biotechnology for Industrial Wasterwater, Vanderbilt University, Archae Press, Nashville, p 115 (1996). The bottles were maintained at a temperature of 35° C.

TABLE 11
Test conditions for ATA and BMP assays

| | Seed | Nutrient | Syn Feed | iBA (3%) | Bicarb Sol. | Water | |
|---|---|---|---|---|---|---|---|
| Test Condition | (mL) | Broth (mL) | (mL) | (mL) | (mL) | (mL) | Total |
| Feed Control | 25 | 84 | 36 | 0 | 40 | 215 | 400 |
| 2X Feed Control | 25 | 84 | 71 | 0 | 40 | 180 | 400 |
| ATA-syn 550 ppm iBA | 25 | 84 | 36 | 7 | 40 | 208 | 400 |
| ATA-syn 825 ppm iBA | 25 | 84 | 36 | 11 | 40 | 204 | 400 |
| ATA-syn 1100 ppm iBA | 25 | 84 | 36 | 15 | 40 | 200 | 400 |
| BMP 550 ppm iBA | 25 | 84 | 0 | 7 | 40 | 244 | 400 |
| BMP 1100 ppm iBA | 25 | 84 | 0 | 15 | 40 | 236 | 400 |
| BMP 1650 ppm iBA | 25 | 84 | 0 | 22 | 40 | 229 | 400 |
| BMP 2200 ppm iBA | 25 | 84 | 0 | 29 | 40 | 222 | 400 |

The results of the BMP tests demonstrated that iBA was biodegradable, though the anaerobic microorganisms appeared to require acclimation to iBA as evidenced by the increasing lag time in gas production with increasing iBA concentrations as shown in Table 12. However, the BMP tests showed a high degree of mineralization within a short time period as evidenced by the generation of 70-80% of their predicted theoretical gas production. The degradability of iBA was further demonstrated by the total organic carbon (TOC) and chemical oxygen demand (COD) removals evidenced by pre- and post-test characterizations of the BMP tests (Table 13).

TABLE 12
Isobutyric acid degradability

| | Cumulative Gas Production (% Theoretical Max) | | | | | | | | | | |
|---|---|---|---|---|---|---|---|---|---|---|---|
| | Day | | | | | | | | | | |
| Test Cond | 2 | 4 | 6 | 8 | 10 | 11 | 12 | 13 | 14 | 15 | 16 |
| Feed Cont. | 15 | 27 | 39 | 48 | ND | 65 | ND | 79 | ND | ND | 86 |
| 2X Feed Cont. | 4 | 10 | 15 | 20 | ND | 33 | ND | 44 | ND | ND | 54 |
| BMP 550 ppm iBA | 19 | 48 | 69 | ND | 73 | ND | 74 | ND | ND | 76 | ND |
| BMP 1100 ppm iBA | 9 | 29 | 69 | ND | 79 | ND | 80 | ND | ND | 81 | ND |
| BMP 1650 ppm iBA | 7 | 18 | 50 | ND | 73 | ND | 74 | ND | ND | 76 | ND |
| BMP 2200 ppm iBA | 4 | 10 | 25 | ND | 68 | ND | 71 | ND | ND | 72 | ND |
| ATA-syn 550 ppm iBA | 18 | 45 | 65 | ND | 82 | ND | 85 | ND | ND | 86 | ND |
| ATA-syn 825 ppm iBA | 14 | 36 | 56 | ND | 82 | ND | 83 | ND | ND | 84 | ND |
| ATA-syn 1100 ppm iBA | 14 | 36 | 51 | ND | 82 | ND | 85 | ND | ND | 85 | ND |

(ND = not determined)

TABLE 13
Organic removal table.

| Test Case | TOC removed | COD removed |
|---|---|---|
| Feed Control | 85% | 95% |
| 2X Feed Control | 97% | 97% |
| ATA-syn + 550 ppm iBA | 94% | 98% |
| ATA-syn + 825 ppm iBA | 95% | 97% |
| ATA-syn + 1100 ppm iBA | 96% | 98% |
| BMP 550 ppm iBA | 85% | 92% |
| BMP 1100 ppm iBA | 97% | 97% |
| BMP 1650 ppm iBA | 92% | 98% |
| BMP 2200 ppm iBA | 97% | 98% |

The ATA tests additionally demonstrated that iBA concentrations of up to 1100 parts per million (ppm) did not inhibit gas generation, as shown by the gas generation rates of the iBA dosed cases by comparisons to the control (Table 12). The organic removal rates were also positive, as shown in Table 13.

### Example 7

### Reduction in Isobutyric Acid Activity by Ion Exchange-Based Chromatography

To determine if isobutyric acid is capable of being removed from a composition comprising isobutanol, ion exchange-based chromatography was performed. A chromatography column was set up as a closed system with one inlet on top of the column (inner diameter: 2.8 cm, length: 13.2 cm) and one outlet at the bottom of the column. The inlet on the top of the column was connected with switchable supply reservoirs by a tube (Masterflex silicone size 14, Cole-Parmer, Vernon Hills, Ill.), and flow from the reservoirs was controlled by a first pump (pump 1) (MasterFlex L/S 77201-60, Cole-Parmer, Vernon Hills, Ill.). The outlet was connected by a tube (Masterflex silicone size 14, Cole-Parmer) with a fraction collector (Bio-RAD model 2110 fraction collector, Bio-Rad Laboratories, Hercules, Calif.), and the flow controlled by a second pump (pump 2) (MasterFlex L/S 77201-60, Cole-Parmer). At the bottom of the column there was a frit, preventing the Diaion WA-30 (Supelco, Sigma Aldrich; St. Louis, Mo.) resin to enter the outlet. In total, 4.0854 g dry weight of Diaion WA-30 was filled into the column.

The experimental sequence was initiated by conditioning the resin. Isobutanol was pumped into the column via pump 1 until the resin was immersed. Subsequently, the liquid in the column was completely drained by pump 2. Next an isobutanol solution containing 100 mg/l isobutyric acid was fed into the column at a pump speed of 0.6 ml/min via pump 1 until the resin was immersed marking the start of the experiment. Then pump 2, the outlet pump, was started at a pump rate of 0.6 ml/min and the fraction collector started to collect about 100 drops per tube. The tubes were weighed before and after sample collection in order to get a precise determination of the collected volume. After pumping about 30 ml, pump 1 was stopped. Pump 2 was operated until all the isobutanol/isobutyric acid solution was drained from the resin. Next a washing step with isobutanol was carried out through filling the column with pure isobutanol via pump 1 at a pump rate of 0.6 ml/min until all the resin was covered. Pump 1 was stopped and pump 2 was turned on at a rate of 0.6 ml/min to remove the isobutanol from the column. The washing steps described with isobutanol were repeated with ethanol. After washing, a 0.5 M NaOH solution was pumped at a rate of 0.6 ml/min via pump 1 onto the column. After the regeneration step, the column was drained of the liquid by stopping pump 1 and operating pump 2 at a rate of 0.6 ml/min. Afterwards an ethanol and then an isobutanol washing step was performed similar to the previous wash steps but with less volume.

Eppendorf tubes in the fraction collector were weighted before and after collecting eluent. Subsequently the weight increase was used to calculate the eluted volume by assuming a density of isobutanol, ethanol and NaOH of 0.802 g/ml, 0.789 g/ml and 1.000 g/ml, respectively. Collected samples were analyzed by gas chromatography coupled to a flame ionization detector (GC-FID) and high-pressure liquid chromatography and ultraviolet (λ=210 nm) detector (HPLC-UV) in order to determine isobutyric acid concentration. For GC-FID analysis, a HP6890 series GC system with a 7683 series injector and coupled to a flame ionization detector was used (Agilent, Wilmington, Del.). As column a Restek stabilwax (Restek, Bellefonte, Pa.) was applied, 30 m×0.53 mm×1 μm, operated with a constant Helium flow of 5.0 ml/min. Injector was set to 240° C. with a split-ratio of 1:25 receiving an injection volume of 1 μl. An oven was tempered at 45° C. for 1.0 min, ramped up to 210° C. at 10° C./min, then ramped to 240° C. at 40° C./min and held at 240° C. for 7.25 mins. Calibration was accomplished with isobutyric acid standards dissolved in isobutanol. Retention time (RT) of isobutyric acid was about 12.3 min. For HPLC-UV analysis, an Aminex® HPX-87H column (Bio-Rad, Hercules, Calif.) was used in an isocratic method with 0.01N sulfuric acid as eluent on an Alliance® 2695 Separations Module (Waters Corp., Milford, Mass.). Flow rate was 0.60 mL/min, column temperature 40° C., injection volume 10 μL and run time 58 min. Detection was carried out with an UV detector (Waters 2996 PDA, Waters Corp., Milford, Mass.) at 210 nm.

The results of the experiment are shown in Table 14. Isobutyric acid was only detected in the 0.5M NaOH eluent. In total, about 3302 mg of isobutyric acid were adsorbed with the resin in the experiment and about 2045 mg of isobutyric acid (>60%) were recovered in the eluent stream. The results indicate that low concentrations of isobutyric acid in isobutanol can be efficiently reduced by ion exchange chromatography and that ion exchange chromatography can be used to enrich isobutyric acid from an isobutyric acid/isobutanol mixture, as indicated by the increased isobutyric acid concentrations in the NaOH eluent stream. Further, the anion exchange resin, Diaion WA-30, can be regenerated by using aqueous 0.5M NaOH solution. Additionally, while isobutanol is miscible with aqueous concentrations up to about 88 g/L, at higher concentrations isobutanol and aqueous solutions form separate phases. In order to allow for efficient and homologous extraction of isobutyric acid from isobutanol by an ion exchange resin, as well as efficient regeneration of the ion exchange resin with aqueous acidic or caustic solutions, compounds soluble at high concentrations in both phases (e.g., ethanol in present example) were used in the intermediary washing steps.

TABLE 14
Samples collected in the fraction collector and the corresponding feed
into the ion exchange column. Vacc indicates the total accumulated
eluent volume of the experiment. The HPLC and GC column indicate concentrations
measured by the described HPLC-UV and GC-FID methods.

| | | w(tube + | d(eluent) | V(eluent) | V_{acc} | HPLC | GC |
|---|---|---|---|---|---|---|---|
| sample | feed | eluent) (g) | (g/ml) | (ml) | (ml) | (mg/l) | (mg/l) |
| T1 | iso + iba | 1.915 | 0.802 | 1.151 | 1.151 | << | << |
| T2 | iso + iba | 1.915 | 0.802 | 1.149 | 2.300 | << | << |
| T3 | iso + iba | 1.914 | 0.802 | 1.148 | 3.448 | << | << |
| T4 | iso + iba | 1.914 | 0.802 | 1.150 | 4.597 | << | << |
| T5 | iso + iba | 1.917 | 0.802 | 1.148 | 5.746 | << | << |
| T6 | iso + iba | 1.915 | 0.802 | 1.147 | 6.893 | << | << |
| T7 | iso + iba | 1.921 | 0.802 | 1.150 | 8.043 | << | << |
| T8 | iso + iba | 1.924 | 0.802 | 1.149 | 9.192 | << | << |
| T9 | iso + iba | 1.924 | 0.802 | 1.149 | 10.341 | << | << |
| T10 | iso + iba | 1.913 | 0.802 | 1.145 | 11.485 | << | << |
| T11 | iso + iba | 1.911 | 0.802 | 1.145 | 12.631 | << | << |
| T12 | iso + iba | 1.915 | 0.802 | 1.147 | 13.777 | << | << |
| T13 | iso + iba | 1.911 | 0.802 | 1.145 | 14.922 | << | << |
| T14 | iso + iba | 1.907 | 0.802 | 1.146 | 16.067 | << | << |
| T15 | iso + iba | 1.912 | 0.802 | 1.146 | 17.213 | << | << |
| T16 | iso + iba | 1.892 | 0.802 | 1.121 | 18.334 | << | << |
| T17 | iso + iba | 1.915 | 0.802 | 1.144 | 19.477 | << | << |
| T18 | iso + iba | 1.912 | 0.802 | 1.145 | 20.623 | << | << |
| T19 | iso + iba | 1.915 | 0.802 | 1.146 | 21.768 | << | << |
| T20 | iso + iba | 1.910 | 0.802 | 1.146 | 22.914 | << | << |
| T21 | iso + iba | 1.913 | 0.802 | 1.144 | 24.058 | << | << |
| T22 | iso + iba | 1.904 | 0.802 | 1.142 | 25.200 | << | << |
| T23 | iso + iba | 1.906 | 0.802 | 1.142 | 26.342 | << | << |
| T24 | iso + iba | 1.889 | 0.802 | 1.107 | 27.449 | << | << |
| T25 | iso + iba | 1.887 | 0.802 | 1.123 | 28.572 | << | << |
| T27 | iso + iba | 1.851 | 0.802 | 1.060 | 29.632 | << | << |
| T28 | iso + iba | 1.887 | 0.802 | 1.108 | 30.740 | << | << |
| T29 | iso + iba | 1.907 | 0.802 | 1.137 | 31.877 | << | << |
| T30 | iso + iba | 1.915 | 0.802 | 1.145 | 33.022 | << | << |
| T31 | no | 1.909 | 0.802 | 1.141 | 34.163 | << | << |
| T32 | no | 1.911 | 0.802 | 1.141 | 35.305 | << | << |
| T33 | no | 1.911 | 0.802 | 1.144 | 36.448 | << | << |
| T34 | no | 1.911 | 0.802 | 1.143 | 37.591 | << | << |
| T35 | no | 1.906 | 0.802 | 1.142 | 38.733 | << | << |
| T36 | no | 1.907 | 0.802 | 1.142 | 39.875 | << | << |
| T37 | no | 1.103 | 0.802 | 0.140 | 40.015 | << | << |
| T38 | iso | 1.905 | 0.802 | 1.134 | 41.149 | << | << |
| T39 | iso | 1.880 | 0.802 | 1.104 | 42.253 | << | << |
| T40 | iso | 1.827 | 0.802 | 1.039 | 43.292 | << | << |
| T41 | iso | 1.948 | 0.802 | 1.194 | 44.486 | << | << |
| T42 | iso | 1.925 | 0.802 | 1.165 | 45.650 | << | << |
| T43 | iso | 1.864 | 0.802 | 1.077 | 46.727 | << | << |
| T44 | iso | 1.938 | 0.802 | 1.173 | 47.900 | << | << |
| T45 | iso | 1.771 | 0.802 | 0.954 | 48.854 | << | << |
| T46 | ethanol | 1.917 | 0.789 | 1.170 | 50.024 | << | << |
| T47 | ethanol | 1.917 | 0.789 | 1.168 | 51.192 | << | << |
| T48 | ethanol | 1.908 | 0.789 | 1.161 | 52.353 | << | << |
| T49 | ethanol | 1.913 | 0.789 | 1.165 | 53.518 | << | << |
| T50 | ethanol | 1.925 | 0.789 | 1.169 | 54.687 | << | << |
| T51 | ethanol | 1.218 | 0.789 | 0.291 | 54.978 | << | << |
| T52 | NaOH | 1.938 | 1.000 | 0.938 | 55.915 | 100 | 92 |
| T53 | NaOH | 1.973 | 1.000 | 0.975 | 56.890 | 140 | 94 |
| T54 | NaOH | 1.992 | 1.000 | 0.999 | 57.889 | 185 | 141 |
| T55 | NaOH | 2.023 | 1.000 | 1.029 | 58.918 | 191 | 145 |
| T56 | NaOH | 2.023 | 1.000 | 1.030 | 59.948 | 193 | 140 |
| T57 | NaOH | 2.199 | 1.000 | 1.202 | 61.150 | 151 | 124 |
| T58 | NaOH | 2.250 | 1.000 | 1.254 | 62.404 | 156 | 128 |
| T59 | NaOH | 2.256 | 1.000 | 1.263 | 63.667 | 160 | 132 |
| T60 | NaOH | 2.213 | 1.000 | 1.222 | 64.889 | 168 | 125 |
| T61 | NaOH | 2.239 | 1.000 | 1.241 | 66.130 | 123 | 106 |
| T62 | NaOH | 2.368 | 1.000 | 1.376 | 67.506 | 95 | 90 |
| T63 | NaOH | 2.482 | 1.000 | 1.487 | 68.993 | 84 | 85 |
| T64 | NaOH | 1.475 | 1.000 | 0.487 | 69.480 | 91 | 86 |
| T65 | ethanol | 2.199 | 0.789 | 1.525 | 71.005 | << | << |
| T66 | ethanol | 2.063 | 0.789 | 1.353 | 72.358 | << | << |
| T67 | ethanol | 1.897 | 0.789 | 1.152 | 73.511 | << | << |
| T68 | ethanol | 1.364 | 0.789 | 0.468 | 73.979 | << | << |
| T69 | iso | 1.911 | 0.802 | 1.140 | 75.119 | << | << |
| T70 | iso | 1.942 | 0.802 | 1.183 | 76.303 | << | << |
| T71 | iso | 1.939 | 0.802 | 1.179 | 77.482 | << | << |
| T72 | iso | 1.634 | 0.802 | 0.797 | 78.278 | << | << |

### Example 8

### Reduction in Carboxylic Acid Activity by Ion Exchange-Based Adsorption

To determine if carboxylic acid mixtures are capable of being removed from a composition comprising isobutanol, ion exchange-based adsorption was performed. Adsorption kinetics of isobutyric acid and acetic acid mixtures in isobutanol to the resins Amberlite IRA-67 (IRA67) (Fluka, Sigma Aldrich), Amberlite IRA-96 (IRA96) (Fluka, Sigma Aldrich) and Diaion WA30 (WA30) (Supelco, Sigma Aldrich) was characterized at 10° C. and 40° C. Approximately 0.5 grams of dry weight of respective resin were added into 1.7 ml Eppendorf tubes. The weight of the Eppendorf tube before and after addition of the resin was determined, designated w(tube) and w(tube+resin), respectively, in order to determine the exact amount of dry resin added, w(dry resin) (Table 15). Subsequently 0.8 ml of pure isobutanol was added to each Eppendorf tube with resin, the Eppendorf tube was closed, vortexed and incubated for approximately one hour at room temperature. Next, the solutions in the Eppendorf tubes were centrifuged and accessible isobutanol was removed by pipette. Subsequently 0.6 ml of an isobutanol solution containing approximately 1, 5, 10, 20 and 30 g/l of each isobutyric and acetic acid was added to each tube. The exact concentration of the added standard solution was measured by HPLC and was designated cₛ(acid). After addition, the weight of the Eppendorf tubes was determined again, yielding w(loaded). The total solvent volume in each tube was calculated from the weight difference between w(loaded) and w(tube+resin), assuming a uniform density of the solutions of 0.802 g/cm³. The tubes containing solutions with different carboxylic acid concentrations and different resins were mixed at room temperature for 4 hours. Finally, samples of the isobutanol solutions in the tubes were withdrawn and analyzed by high performance liquid chromatography (HPLC), yielding c_{eq}(acid) of isobutyric and acetic acid, respectively. The results at 10° C. and 40° C. are shown in Tables 16 and 17, respectively.

TABLE 15
Equlibrium experiments with resins and mixtures of isobutyric and acetic
acid dissolved in isobutanol at 10° C. and 40° C.

| | | Temp | W(tube + | W(dry | W(loaded) | Vₜₒₜₐₗ |
|---|---|---|---|---|---|---|
| Resin | Acid | (° C.) | resin) (g) | resin) (g) | (g) | (ml) |
| WA30 | IBA + AC | 10 | 1.488 | 0.499 | 2.342 | 1.065 |
| WA30 | IBA + AC | 10 | 1.486 | 0.502 | 2.341 | 1.067 |
| WA30 | IBA + AC | 10 | 1.491 | 0.500 | 2.347 | 1.067 |
| WA30 | IBA + AC | 10 | 1.490 | 0.500 | 2.337 | 1.056 |
| WA30 | IBA + AC | 10 | 1.485 | 0.503 | 2.346 | 1.073 |
| IRA67 | IBA + AC | 10 | 1.484 | 0.503 | 2.232 | 0.932 |
| IRA67 | IBA + AC | 10 | 1.480 | 0.500 | 2.232 | 0.937 |
| IRA67 | IBA + AC | 10 | 1.493 | 0.502 | 2.259 | 0.955 |
| IRA67 | IBA + AC | 10 | 1.484 | 0.503 | 2.228 | 0.928 |
| IRA67 | IBA + AC | 10 | 1.491 | 0.500 | 2.246 | 0.941 |
| IRA96 | IBA + AC | 10 | 1.490 | 0.501 | 2.286 | 0.992 |
| IRA96 | IBA + AC | 10 | 1.480 | 0.500 | 2.285 | 1.003 |
| IRA96 | IBA + AC | 10 | 1.484 | 0.499 | 2.276 | 0.988 |
| IRA96 | IBA + AC | 10 | 1.488 | 0.503 | 2.304 | 1.017 |
| IRA96 | IBA + AC | 10 | 1.494 | 0.504 | 2.310 | 1.017 |
| WA30 | IBA + AC | 40 | 1.489 | 0.500 | 2.272 | 0.976 |
| WA30 | IBA + AC | 40 | 1.488 | 0.496 | 2.292 | 1.003 |
| WA30 | IBA + AC | 40 | 1.482 | 0.498 | 2.297 | 1.016 |
| WA30 | IBA + AC | 40 | 1.481 | 0.494 | 2.312 | 1.036 |
| WA30 | IBA + AC | 40 | 1.486 | 0.498 | 2.262 | 0.967 |
| IRA67 | IBA + AC | 40 | 1.491 | 0.500 | 2.118 | 0.781 |
| IRA67 | IBA + AC | 40 | 1.481 | 0.497 | 2.161 | 0.847 |
| IRA67 | IBA + AC | 40 | 1.484 | 0.496 | 2.208 | 0.902 |
| IRA67 | IBA + AC | 40 | 1.481 | 0.497 | 2.229 | 0.933 |
| IRA67 | IBA + AC | 40 | 1.474 | 0.492 | 2.189 | 0.891 |
| IRA96 | IBA + AC | 40 | 1.494 | 0.508 | 2.265 | 0.962 |
| IRA96 | IBA + AC | 40 | 1.487 | 0.497 | 2.289 | 0.999 |
| IRA96 | IBA + AC | 40 | 1.492 | 0.500 | 2.214 | 0.901 |
| IRA96 | IBA + AC | 40 | 1.494 | 0.502 | 2.256 | 0.951 |
| IRA96 | IBA + AC | 40 | 1.488 | 0.499 | 2.224 | 0.917 |

IBA = isobutyric acid, AC = acetic acid.

TABLE 16
Equilibrium experiments with resins and mixtures of isobutyric and acetic acid dissolved in isobutanol at 10° C.

| | | C₅(IBA) | C₅(AC) | C_{load} (IBA) | C_{load} (AC) | C_{eq}(IBA) | C_{eq}(AC) | N_{ad}(IBA) | N_{ad}(AC) |
|---|---|---|---|---|---|---|---|---|---|
| Resin | Acid | (mM) | (mM) | (mM) | (mM) | (mM) | (mM) | (mmol/g) | (mmol/g) |
| WA30 | IBA + AC | 8.5 | 12.7 | 4.8 | 7.1 | 0.0 | 0.0 | 0.010 | 0.015 |
| WA30 | IBA + AC | 66.4 | 84.4 | 37.3 | 47.5 | 2.8 | 1.1 | 0.073 | 0.099 |
| WA30 | IBA + AC | 118.4 | 178.7 | 66.5 | 100.4 | 7.7 | 2.9 | 0.126 | 0.208 |
| WA30 | IBA + AC | 220.9 | 354.0 | 125.5 | 201.2 | 29.7 | 10.0 | 0.202 | 0.403 |
| WA30 | IBA + AC | 352.4 | 479.6 | 197.1 | 268.2 | 32.7 | 8.4 | 0.350 | 0.554 |
| IRA67 | IBA + AC | 8.5 | 12.7 | 5.4 | 8.2 | 1.2 | 1.1 | 0.008 | 0.013 |
| IRA67 | IBA + AC | 66.4 | 84.4 | 42.5 | 54.0 | 3.4 | 1.9 | 0.073 | 0.098 |
| IRA67 | IBA + AC | 118.4 | 178.7 | 74.4 | 112.3 | 4.9 | 2.1 | 0.132 | 0.209 |
| IRA67 | IBA + AC | 220.9 | 354.0 | 142.8 | 228.8 | 43.5 | 9.1 | 0.183 | 0.405 |
| IRA67 | IBA + AC | 352.4 | 479.6 | 224.6 | 305.7 | 86.6 | 15.2 | 0.260 | 0.547 |
| IRA96 | IBA + AC | 8.5 | 12.7 | 5.1 | 7.7 | 1.7 | 1.5 | 0.007 | 0.012 |
| IRA96 | IBA + AC | 66.4 | 84.4 | 39.7 | 50.5 | 14.2 | 7.7 | 0.051 | 0.086 |
| IRA96 | IBA + AC | 118.4 | 178.7 | 71.9 | 108.5 | 26.5 | 13.1 | 0.090 | 0.189 |
| IRA96 | IBA + AC | 220.9 | 354.0 | 130.3 | 208.9 | 63.3 | 26.4 | 0.135 | 0.369 |
| IRA96 | IBA + AC | 352.4 | 479.6 | 207.8 | 282.8 | 157.2 | 69.7 | 0.102 | 0.431 |

IBA = isobutyric acid, AC = acetic acid.

TABLE 17
Equilibrium experiments with resins and mixtures of isobutyric and acetic acid dissolved in isobutanol at 40° C.

| | | C₅(IBA) | C₅(AC) | C_{load} (IBA) | C_{load} (AC) | C_{eq}(IBA) | C_{eq}(AC) | N_{ad}(IBA) | N_{ad}(AC) |
|---|---|---|---|---|---|---|---|---|---|
| Resin | Acid | (mM) | (mM) | (mM) | (mM) | (mM) | (mM) | (mmol/g) | (mmol/g) |
| WA30 | IBA + AC | 8.5 | 12.7 | 5.2 | 7.8 | 0.0 | 0.0 | 0.010 | 0.015 |
| WA30 | IBA + AC | 66.4 | 84.4 | 39.7 | 50.5 | 2.7 | 1.3 | 0.075 | 0.099 |
| WA30 | IBA + AC | 118.4 | 178.7 | 69.9 | 105.5 | 5.2 | 2.4 | 0.132 | 0.210 |
| WA30 | IBA + AC | 220.9 | 354.0 | 127.9 | 205.0 | 18.6 | 7.3 | 0.229 | 0.415 |
| WA30 | IBA + AC | 352.4 | 479.6 | 218.7 | 297.6 | 56.4 | 17.8 | 0.315 | 0.544 |
| IRA67 | IBA + AC | 8.5 | 12.7 | 6.5 | 9.7 | 1.1 | 1.2 | 0.008 | 0.013 |
| IRA67 | IBA + AC | 66.4 | 84.4 | 47.0 | 59.8 | 3.4 | 2.2 | 0.074 | 0.098 |
| IRA67 | IBA + AC | 118.4 | 178.7 | 78.7 | 118.8 | 3.7 | 2.8 | 0.137 | 0.211 |
| IRA67 | IBA + AC | 220.9 | 354.0 | 142.1 | 227.7 | 7.9 | 3.4 | 0.252 | 0.421 |
| IRA67 | IBA + AC | 352.4 | 479.6 | 237.4 | 323.1 | 9.9 | 3.6 | 0.412 | 0.578 |
| IRA96 | IBA + AC | 8.5 | 12.7 | 5.3 | 7.9 | 2.0 | 1.7 | 0.006 | 0.012 |
| IRA96 | IBA + AC | 66.4 | 84.4 | 39.9 | 50.7 | 9.3 | 4.8 | 0.061 | 0.092 |
| IRA96 | IBA + AC | 118.4 | 178.7 | 78.8 | 118.9 | 14.6 | 8.5 | 0.116 | 0.199 |
| IRA96 | IBA + AC | 220.9 | 354.0 | 139.4 | 223.4 | 48.4 | 26.7 | 0.172 | 0.373 |
| IRA96 | IBA + AC | 352.4 | 479.6 | 230.5 | 313.8 | 80.8 | 29.8 | 0.275 | 0.522 |

IBA = isobutyric acid, AC = acetic acid.

The adsorbed amounts of respective acids, n_{ad}(IBA) and n_{ad}(ACA), were calculated and compared to Langmuir adsorption models that were obtained from analysis of each of the single acids dissolved in isobutanol at room temperature (RT). Parameters of the used single acid adsorption kinetics are provided in Table 18. Measured data and previously determined single-substrate Langmuir adsorption isotherms are depicted in Table 19.

TABLE 18
Fitted Langmuir adsorption isotherms for IRA67 and WA30 resins for
isobutyric acid or acetic acid in isobutanol at room temperature.

| | | | Iₘₐₓ (spec) | |
|---|---|---|---|---|
| Resin | Carboxylic Acid | Iₘₐₓ (mmol/g) | (mmol/g) | α[mM⁻¹] |
| IRA67 | Isobutyric | 0.959 | n.a. | 0.006 |
| WA30 | Isobutyric | 0.761 | 3.000 | 0.008 |
| IRA67 | Acetic | 1.621 | n.a. | 0.007 |
| WA30 | Acetic | 2.314 | 3.000 | 0.006 |

TABLE 19
Measured adsorbed isobutyric acid or acetic acid versus measured predicted values.

| | Load | | Solvent | C₍ₜ₌₀₎ | C₍ₜ₌₄₎ | Dry | Nad (meas) | Nad (pred) |
|---|---|---|---|---|---|---|---|---|
| Sample | (ml) | Cᵢₙᵢₜ (mM) | (ml) | (mM) | (mM) | resin (g) | (mmol/g) | (mmol/g) |
| IC-IRA67-IBA-1 | 0.60 | 11.9 | 0.931 | 7.7 | 2.4 | 0.501 | 0.010 | 0.013 |
| IC-IRA67-IBA-5 | 0.60 | 57.5 | 0.884 | 39.0 | 5.4 | 0.498 | 0.060 | 0.028 |
| IC-IRA67-IBA-10 | 0.60 | 115.9 | 0.946 | 73.5 | 10.0 | 0.500 | 0.120 | 0.051 |
| IC-IRA67-IBA-20 | 0.60 | 231.5 | 0.944 | 147.1 | 49.3 | 0.505 | 0.183 | 0.209 |
| IC-IRA67-IBA-30 | 0.60 | 344.0 | 0.945 | 218.4 | 64.0 | 0.499 | 0.293 | 0.255 |
| IC-IRA67-IBA-50 | 0.60 | 570.7 | 0.913 | 375.0 | 152.1 | 0.503 | 0.405 | 0.444 |
| IC-IRA67-IBA-100 | 0.60 | 1122.3 | 0.936 | 719.2 | 352.7 | 0.503 | 0.682 | 0.639 |
| IC-IRA67-AC-1 | 0.60 | 22 | 0.939 | 13.9 | 2.3 | 0.498 | 0.022 | 0.025 |
| IC-IRA67-AC-10 | 0.60 | 167 | 0.935 | 107.5 | 4.7 | 0.505 | 0.190 | 0.050 |
| IC-IRA67-AC-20 | 0.60 | 328 | 0.936 | 210.0 | 11.2 | 0.504 | 0.369 | 0.114 |
| IC-IRA67-AC-40 | 0.60 | 658 | 0.928 | 425.7 | 133.7 | 0.502 | 0.540 | 0.769 |
| IC-IRA67-AC-60 | 0.60 | 973 | 0.933 | 625.3 | 134.2 | 0.500 | 0.918 | 0.770 |
| IC-IRA67-AC-100 | 0.60 | 1616 | 0.958 | 1011.9 | 397.5 | 0.497 | 1.184 | 1.181 |
| IC-IRA67-AC-200 | 0.60 | 3129 | 0.955 | 1965.3 | 782.1 | 0.498 | 2.272 | 1.363 |
| IC-WA30-IBA-1 | 0.60 | 11.9 | 1.026 | 7.0 | 0.5 | 0.497 | 0.013 | 0.003 |
| IC-WA30-IBA-5 | 0.60 | 57.5 | 1.064 | 32.4 | 5.0 | 0.500 | 0.058 | 0.030 |
| IC-WA30-IBA-10 | 0.60 | 115.9 | 1.057 | 65.8 | 22.6 | 0.497 | 0.092 | 0.119 |
| IC-WA30-IBA-20 | 0.60 | 231.5 | 1.048 | 132.5 | 41.4 | 0.502 | 0.190 | 0.193 |
| IC-WA30-IBA-30 | 0.60 | 344.0 | 1.059 | 194.9 | 82.2 | 0.504 | 0.237 | 0.306 |
| IC-WA30-IBA-50 | 0.60 | 570.7 | 1.039 | 329.7 | 107.7 | 0.501 | 0.460 | 0.357 |
| IC-WA30-IBA-100 | 0.60 | 1122.3 | 1.037 | 649.7 | 306.8 | 0.510 | 0.697 | 0.544 |
| IC-WA30-AC-1 | 0.60 | 22 | 1.048 | 12.5 | 0.2 | 0.500 | 0.026 | 0.002 |
| IC-WA30-AC-10 | 0.60 | 167 | 1.041 | 96.4 | 15.3 | 0.501 | 0.169 | 0.192 |
| IC-WA30-AC-20 | 0.60 | 328 | 1.021 | 192.5 | 25.3 | 0.502 | 0.340 | 0.301 |
| IC-WA30-AC-40 | 0.60 | 658 | 1.035 | 381.7 | 61.5 | 0.504 | 0.657 | 0.617 |
| IC-WA30-AC-60 | 0.60 | 973 | 1.038 | 562.5 | 126.1 | 0.497 | 0.912 | 0.988 |
| IC-WA30-AC-100 | 0.60 | 1616 | 1.058 | 916.1 | 284.1 | 0.501 | 1.334 | 1.450 |
| IC-WA30-AC-200 | 0.60 | 3129 | 1.097 | 1711.3 | 743.7 | 0.499 | 2.127 | 1.885 |

IBA = isobutyric acid, AC = acetic acid

The results indicate that ion exchange-based chromatography reduced isobutyric and acetic acid concentrations in isobutanol compositions. Weak basic anion exchange resins, such as WA30, IRA67, and IRA96 were capable of reducing or removing isobutyric and acetic acid from isobutanol compositions comprising isobutyric and acetic acid with the order of favorable adsorption kinetics for the investigated resins for carboxylic acids in isobutanol being WA30>IRA67>IRA96. Further, adsorption at lower concentrations in carboxylic acid mixtures in isobutanol is more effective than single compound adsorption, as evidenced by the higher adsorption than predicted by the determined Langmuir isotherm kinetics. Additionally, as evidenced by Tables 16 and 17, increased temperature resulted in increased adsorption by the ion exchange resins.

### Example 8

### Regeneration of Ion Exchange Resins after Adsorption of Carboxylic Acids

Regeneration refers to the process of returning the stationary phase of an ion exchange resin to its initial state after performing the ion exchange process. Regeneration involves replacing ions taken up in the exchange process with the desired ions that occupied the exchange sites at the beginning of the ion exchange process. To determine if ion exchange resins could be regenerated after adsorption of carboxylic acids (e.g., isobutyric and/or acetic acid), the use of acidic or caustic solutions was evaluated. As an exemplary acidic solution aqueous 1.0 M HCl was chosen, and for an exemplary caustic solution aqueous 0.5 M NaOH was chosen. Employed ion exchange resins were either Amberlite IRA-67 (IRA67) (FLUKA, Sigma Aldrich) or Diaion WA-30 (WA30) (SUPELCO, Sigma Aldrich). Samples from Example 8 were used as a starting point. Briefly, these samples were provided in 2.0 ml Eppendorf tubes which contained one of the two respective ion exchange resins of determined weight in equilibrium with butanol solutions containing either isobutyric or acetic acid of known concentration. Comparable to adsorption isotherms, equilibrium concentrations of solutions were determined for assessing suitability of the acid or caustic solution for regeneration.

Initially, all of equilibrated isobutanol-acid solutions were pipetted out of the samples and the weight of the reminder wet resin with adsorbed isobutyric or acetic acid was determined. Based on the knowledge from the previous experiment of the weight of the dry resin, the weight of the Eppendorf tube with dry resin as well as the concentrations of the respective acid in the samples, and assuming a density of 0.802 g/cm³ for the reminder isobutanol solution, the amounts of adsorbed acid to the resin, n(ad), and the amount of respective acid in the residual isobutanol liquid, n(residual) was calculated. Subsequently approximately 0.60+/−0.05 ml of ethanol (proof 200) was added to the wet resin, the mixture well mixed and the weight determined again. Next, the resulting ethanol/butanol solution with either isobutyric or acetic acid was pipetted out, the weigth of the wet resin with remainder liquid and tube determined again, and the withdrawn volume of ethanol V(EW) determined in assuming a density of 0.7890 g/cm³. The ethanol/isobutanol mixture was analyzed by HPLC and the acid concentration c(EW) of either isobutyric or acetic acid determined, respectively. Subsequently, the amount of acid removed from the sample by the ethanol wash step, n(EW), was calculated. Next, 0.5 ml of 1.0 M HCl was added to samples 1, 10, 30 and 100 of either IRA67 or WA30 with either IBA or ACA. Alternatively 0.5 ml of 0.5M NaOH was added to samples 5, 20, 50 of either IRA67 or WA30 with either IBA or ACA. Samples with added regenerant were vortexed and stored for either 30 min (HCl) or 4 h (NaOH). After the incubation, regenerant liquid was removed from the samples by pipette. Again weights were determined before and after addition of the extractant, as well as after removal of the extractant. Weight of the removed acid/caustic treatment step was converted into volume (V1) assuming a specific density of 1.000 g/cm³. Concentrations of either isobutyric or acetic acid c(1) in the removed regenerant solution were determined by HPLC, allowing for the calculation of the removed amount of either isobutyric or acetic acid, n(1). The treatment step for regeneration was repeated again as described for step 1. However, the only difference this time was that the incubation time for both regeneration approaches was 1 h, yielding V(2), c(2) and n(2). An overview on the results of the washing as well as two regeneration steps is provided in Table 20.

TABLE 20
Anion exchange resins IRA67 (I67) and WA30 (WA) in equilibrium with different concentrations
of isobutyric (IBA) and acetic acid (AC). Volumes removed after the treatment step: V_{EW}, V₁,
V₂; concentrations of IBA or AC removed as determined by HPLC: c_{EW}, c₁, and c₂; and total amount of
removed IBA or AC: n_{EW}, n₁, and n₂.

| Sample | | n_{(resid)} | n_{(ad)} | V_{EW} | c_{EW} | n_{EW} | V₁ | c₁ | n₁ | V₂ | c₂ | n₂ |
|---|---|---|---|---|---|---|---|---|---|---|---|---|
| (resin-acid) | Regen. | μmol | μmol | ml | mM | μmol | ml | mM | μmol | ml | mM | μmol |
| I67-IBA1 | HCl | 0.7 | 4.9 | 0.635 | 1.2 | 0.8 | 0.371 | 1.0 | 0.4 | 0.457 | 1.9 | 0.9 |
| I67-IBA5 | NaOH | 1.7 | 29.7 | 0.631 | 3.2 | 2.0 | 0.453 | 21.9 | 9.9 | 0.520 | 12.4 | 6.4 |
| I67-IBA10 | HCl | 3.1 | 60.1 | 0.585 | 3.6 | 2.1 | 0.381 | 16.2 | 6.2 | 0.459 | 23.3 | 10.7 |
| I67-IBA20 | NaOH | 16.7 | 92.4 | 0.597 | 6.0 | 3.6 | 0.480 | 46.9 | 22.5 | 0.519 | 36.0 | 18.7 |
| I67-IBA30 | HCl | 21.9 | 145.9 | 0.611 | 8.3 | 5.1 | 0.382 | 42.4 | 16.1 | 0.489 | 53.3 | 26.1 |
| I67-IBA50 | NaOH | 52.3 | 203.5 | 0.647 | 22.5 | 14.6 | 0.470 | 104.8 | 49.3 | 0.517 | 109.3 | 56.6 |
| I67-IBA100 | HCl | 129.1 | 343.2 | 0.587 | 30.2 | 17.7 | 0.353 | 171.0 | 60.3 | 0.466 | 187.8 | 87.5 |
| I67-AC1 | NaOH | 0.8 | 10.9 | 0.651 | 1.4 | 0.9 | 0.392 | 1.8 | 0.7 | 0.440 | 2.0 | 0.9 |
| I67-AC10 | HCl | 1.5 | 96.0 | 0.605 | 3.2 | 1.9 | 0.476 | 63.6 | 30.2 | 0.511 | 37.6 | 19.2 |
| I67-AC20 | NaOH | 3.8 | 186.1 | 0.680 | 4.2 | 2.9 | 0.350 | 30.0 | 10.5 | 0.441 | 70.8 | 31.3 |
| I67-AC40 | HCl | 38.8 | 270.9 | 0.599 | 6.8 | 4.1 | 0.450 | 143.0 | 64.4 | 0.551 | 175.1 | 96.5 |
| I67-AC60 | NaOH | 43.7 | 458.4 | 0.619 | 10.2 | 6.3 | 0.340 | 114.8 | 39.1 | 0.476 | 206.4 | 98.3 |
| I67-AC100 | HCl | 132.0 | 588.5 | 0.600 | 31.9 | 19.1 | 0.425 | 179.2 | 76.1 | 0.528 | 315.0 | 166.5 |
| I67-AC200 | NaOH | 274.6 | 1130.4 | 0.566 | 212.0 | 120 | 0.313 | 698.1 | 218.5 | 0.508 | 513.6 | 260.8 |
| WA-IBA1 | HCl | 0.2 | 6.7 | 0.577 | 0.0 | 0.0 | 0.403 | 1.9 | 0.8 | 0.419 | 1.9 | 0.8 |
| WA-IBA5 | NaOH | 2.3 | 29.1 | 0.602 | 0.6 | 0.4 | 0.433 | 15.0 | 6.5 | 0.524 | 7.8 | 4.1 |
| WA-IBA10 | HCl | 10.6 | 45.6 | 0.564 | 3.7 | 2.1 | 0.406 | 15.8 | 6.4 | 0.436 | 15.2 | 6.6 |
| WA-IBA20 | NaOH | 19.1 | 95.5 | 0.597 | 11.0 | 6.6 | 0.433 | 41.0 | 17.8 | 0.514 | 22.1 | 11.3 |
| WA-IBA30 | HCl | 38.7 | 119.3 | 0.497 | 19.4 | 9.6 | 0.407 | 46.3 | 18.8 | 0.427 | 46.4 | 19.8 |
| WA-IBA50 | NaOH | 53.2 | 230.6 | 0519 | 43.7 | 22.7 | 0.430 | 41.9 | 18.0 | 0.540 | 69.0 | 37.3 |
| WA-IBA100 | HCl | 163.3 | 355.4 | 0.501 | 134.4 | 67.3 | 0.411 | 58.1 | 23.9 | 0.427 | 101.0 | 43.1 |
| WA-AC1 | NaOH | 0.1 | 12.9 | 1.163 | 0.0 | 0.0 | 0.396 | 2.1 | 0.8 | 0.449 | 2.7 | 1.2 |
| WA-AC10 | HCl | 6.9 | 84.5 | 0.528 | 3.8 | 2.0 | 0.407 | 52.7 | 21.5 | 0.564 | 28.6 | 16.2 |
| WA-AC20 | NaOH | 11.7 | 170.8 | 0.534 | 11.7 | 6.3 | 0.395 | 39.8 | 15.7 | 0.445 | 46.7 | 20.8 |
| WA-AC40 | HCl | 31.2 | 331.2 | 0.493 | 25.3 | 12.5 | 0.459 | 241.6 | 110.9 | 0.535 | 149.4 | 80.0 |
| WA-AC60 | NaOH | 63.8 | 452.8 | 0.489 | 64.5 | 31.6 | 0.376 | 119.2 | 44.8 | 0.455 | 206.3 | 93.9 |
| WA-AC100 | HCl | 165.9 | 668.8 | 0.497 | 131.5 | 65.4 | 0.456 | 216.9 | 98.9 | 0.532 | 317.1 | 168.8 |
| WA-AC200 | NaOH | 455.2 | 1061.6 | 0.497 | 272.6 | 135.4 | 0.373 | 389.0 | 145.0 | 0.468 | 486.2 | 227.6 |

The maximum regeneration capacity of 2×0.5 ml of 0.5 M NaOH at a regeneration ratio of 1 would be 500 umol, of 2×0.5 ml of 1.0 M HCl 1000 umol. Due to the more efficient adsorption of acetic acid to the resins, the maximum expected percentage of regenerated resin sample with HCl in case of the 200 mg/l sample is 71% and 66% for IRA67 and WA30, respectively. In case of the NaOH treated samples previously equilibrated at 100 mg/l isobutyric acid in isobutanol, the maximum expected percentage of regenerated resin sample is 69% and 60% for IRA67 and WA30, respectively. From the data presented in Table 19, the percentage of isobutyric or acetic acid released from the initial resin sample after the ethanol washing step and subsequently treatment with two regeneration steps with either 1.0 M HCl or 0.5 M NaOH was calculated. The percentage of regenerated IRA67 resin that had adsorbed isobutyric acid ranged from about 40-60% when regenerated with 0.5 M NaOH and about 25-40% when regenerated with 1.0 M HCl. The percentage of regenerated WA30 resin that had adsorbed isobutyric acid ranged from about 25-38% when regenerated with 0.5 M NaOH and about 20-32% when regenerated with 1.0 M HCl. The percentage of regenerated IRA67 resin that had adsorbed acetic acid ranged from about 38-57% when regenerated with 0.5M NaOH and about 20-42% when regenerated with 1.0 M HCl. The percentage of regenerated WA30 resin that had adsorbed acetic acid ranged from about 40-60% when regenerated with 0.5 M NaOH and about 18-38% when regenerated with 1.0 M HCl. The range of regeneration percentage varied on the initial concentration of the carboxylic acid.

The results indicate that acidic solutions, such as HCl, or caustic solutions, such as NaOH, were capable of regenerating weak basic anion exchange resins, such as WA30 and IRA67 in aqueous solution. Regeneration of weak basic anion exchange resins was more efficient with caustic solutions as evidenced by the better regeneration percentage. Further, isobutyric acid-loaded IRA67 resin was better to regenerate than the isobutyric acid-loaded WA30 resin.

What is claimed:

1. A process comprising:
(a) providing a recombinant microorganism comprising a butanol biosynthetic pathway;
(b) contacting the recombinant microorganism with a fermentation medium whereby butanol is produced and wherein the fermentation medium comprises one or more carboxylic acids;
(c) adjusting the fermentation medium to reduce the activity of the one or more carboxylic acids.

2. The process of claim 1, wherein the carboxylic acid is selected from the group consisting of butyric acid, valeric acid, propanoic acid, formic acid, and acetic acid.

3. (canceled)

4. The process of claim 2, wherein the butyric acid is isobutyric acid.

5. The process of claim 1, wherein reducing the activity of the one or more carboxylic acids includes adjusting the pH, chemically modifying, sequestering, destroying, or forming a complex with the one or more carboxylic acids.

6. The method of claim 5, wherein adjusting the pH includes increasing or decreasing the pH of the fermentation medium.

7. The process of claim 1, wherein adjusting the fermentation medium includes contacting the fermentation medium with an agent.

8. The process of claim 7, wherein the contacting step occurs during the fermentation.

9. The process of claim 1, wherein adjusting the fermentation medium further comprises distilling the fermentation medium.

10. The process of claim 9, wherein the distillation step results in the isolation of the butanol from the composition.

11. The process of claim 9, wherein the contacting step occurs prior to distillation.

12. The process of claim 11, wherein the contacting step occurs in one or more beer wells.

13. The process of claim 9, wherein the contacting step occurs during distillation.

14. (canceled)

15. The process of claim 13, wherein the distillation step comprises at least one distillation column and at least one decanter vessel.

16. The process of claim 15, wherein the agent is added to the at least one decanter vessel.

17. The process of claim 7, wherein the agent is selected from the group consisting of a sequestering agent, a complexation agent, a neutralizing agent, a modifying agent, and a destructive agent.

18. (canceled)

19. The process of claim 17, wherein the neutralizing agent increases the pH of the composition.

20. The process of claim 19, wherein the neutralizing agent is a base.

21. The process of claim 20, wherein the base is selected from the group consisting of calcium oxide, calcium hydroxide, calcium carbonate, sodium hydroxide, sodium carbonate, sodium phosphate, sodium ethoxide, potassium hydroxide, potassium carbonate, potassium phosphate, magnesium hydroxide, ammonium hydroxide, and combinations thereof.

22. The process of claim 17, wherein the neutralizing agent is selected from the group consisting of urea, fatty amines, anhydrous ammonia, and ion exchange resin.

23. (canceled)

24. The process of claim 17, wherein the modifying agent is an esterifying agent.

25. The process of claim 7, wherein the agent decreases the pH of the fermentation medium, whereby a decrease in the pH of the fermentation medium increases the efficiency of an extractant to extract the one or more carboxylic acids from the fermentation medium.

26. The process of claim 25, wherein the agent is an acid.

27. A process comprising:
(a) providing a feed from a fermentation vessel, wherein the feed comprises a composition produced by a recombinant microorganism comprising a butanol biosynthetic pathway, wherein the composition comprises butanol, water, and one or more carboxylic acids; and
(b) adjusting the feed;
wherein adjusting the feed reduces the activity of the one or more carboxylic acids in the composition.

28. The process of claim 27, wherein adjusting the feed comprises contacting the feed with an agent.

29. The process of claim 27, wherein the carboxylic acid is selected from the group consisting of butyric acid, valeric acid, propanoic acid, formic acid, and acetic acid.

30. (canceled)

31. The process of claim 29, wherein the butyric acid is isobutyric acid.

32. The process of claim 27, further comprising a distillation step (c), wherein the distillation step results in the isolation of the butanol from the composition.

33. The process of claim 32, wherein the adjusting step (b) occurs prior to the distillation step (c).

34. The process of claim 33, wherein the adjusting step occurs in one or more beer wells.

35. The process of claim 32, wherein the adjusting step (b) occurs during the distillation step (c).

36. (canceled)

37. The process of claim 25, wherein the distillation step (c) comprises at least one distillation column and at least one decanter vessel.

38. The process of claim 37, wherein the adjusting step (b) occurs in the at least one decanter vessel.

39. The process of claim 28, wherein the agent is selected from the group consisting of a sequestering agent, a complexation agent, a neutralizing agent, a modifying agent, and a destructive agent.

40. (canceled)

41. The process of claim 39, wherein the neutralizing agent increases the pH of the composition.

42. The process of claim 41, wherein the neutralizing agent is a base.

43. The process of claim 42, wherein the base is selected from the group consisting of calcium oxide, calcium hydroxide, calcium carbonate, sodium hydroxide, sodium carbonate, sodium phosphate, sodium ethoxide, potassium hydroxide, potassium carbonate, potassium phosphate, magnesium hydroxide, ammonium hydroxide, and combinations thereof.

44. The process of claim 39, wherein the neutralizing agent is selected from the group consisting of urea, fatty amines, anhydrous ammonia, and ion exchange resin.

45. (canceled)

46. The process of claim 39, wherein the modifying agent is an esterifying agent.

47. The process of claim 28, wherein the agent decreases the pH of the fermentation medium, whereby a decrease in the pH of the fermentation medium increases the efficiency of an extractant to extract the one or more carboxylic acids from the fermentation medium.

48. The process of claim 47, wherein the agent is an acid.

49. A composition produced by the process of claim 1, wherein the composition comprises less than 1 weight percent carboxylic acid.

50. The composition of claim 49, wherein the composition comprises less than 0.10 weight percent carboxylic acid.

51. The composition of claim 50, wherein the composition comprises less than 0.01 weight percent carboxylic acid.

52. The composition of claim 51, wherein the composition comprises less than 0.001 weight percent carboxylic acid.
